SEQUENCE LISTING (4 sequences)

SEQ ID NO: 1 - protein, 446 aa, Artificial Sequence
  V4-C26 hIgG4(S228P) heavy chain
QVQLQESGPGLVKPSDTLSLTCTVSGYSITSDYTWNWIRQTPGKGLEWIGHITYSGSVSYNPSLRSRVTISRDTSKNQFSLKLSSVTAADTATYSCARALYYPWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, Artificial Sequence
  V4-C26 K light chain
EIVLTQSPATLSLSPGERATLSCRASSSVGYIHWYQQKPGQAPRPIIYTTSNRATGIPARFSGSGSGTDNTLTISSLEPEDSAVYYCQQWSSYPPITFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 1338 bp, Artificial Sequence
  Antibody heavy chain cDNA
caggtgcagctgcaggagtccggaccaggactggtgaagccaagcgacaccctgtccctgacctgcacagtgtccggctactctatcacaagcgattatacctggaactggatcaggcagacacctggcaagggactggagtggatcggccacatcacatattctggctccgtcagctacaatccttccctgcggtctagggtgaccatctccagagacacatctaagaatcagttcagcctgaagctgagctcagtgaccgccgctgatacagccacgtacagctgtgctcgcgccctgtactatccctggtattttgacgtgtggggcactggcaccacagtgacagtgtctagcgctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagcctcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 4 - DNA, 642 bp, Artificial Sequence
  Antibody light chain cDNA
gagatcgtgctcacccagagccctgctacactgtccctgtctccaggagagagggctaccctgtcctgcagggccagctcctctgtgggctacatccactggtatcagcagaagccaggacaggctccacggccaattatctacaccacctctaacagagctacaggcatccctgctcgcttcagcggctccggttctggcacagacaataccctgacaatcagctccctggagcccgaggattccgccgtgtactattgtcagcagtggtctagctatccccctatcacctttggcggcggcacaaagctggaggtgaagcgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2024/0279340 A1
(43) Pub. Date: Aug. 22, 2024

(54) CELLS EXPRESSING VISTA ANTIGEN-BINDING MOLECULES

(71) Applicant: Hummingbird Bioscience Pte. Ltd., Singapore (SG)

(72) Inventors: Jerome Douglas Boyd-Kirkup, Singapore (SG); Piers Ingram, Singapore (SG); Dipti Thakkar, Singapore (SG); Siyu Guan, Singapore (SG); Akila Sadasivam, Singapore (SG)

(21) Appl. No.: 18/570,423

(22) PCT Filed: Jun. 13, 2022
(86) PCT No.: PCT/EP2022/066061

§ 371 Date: Dec. 14, 2023

(30) Foreign Application Priority Data
Jun. 14, 2021 (GB) 2108446.2

(51) Int. Cl. C07K 16/28; A61K 39/00; A61P 35/00

(52) U.S. Cl. CPC C07K 16/2827; A61P 35/00; A61K 2039/505; C07K 2317/24; C07K 2317/33; C07K 2317/73; C07K 2317/92; C07K 2317/94

(57) ABSTRACT

Cells expressing VISTA antigen-binding molecules are disclosed. Also disclosed are compositions comprising such cells, methods for producing VISTA antigen-binding molecules using such cells, and compositions comprising and methods using the VISTA antigen-binding molecules expressed by such cells.

This application claims priority from GB 2108446.2 filed 14 Jun. 2021, the contents and elements of which are herein incorporated by reference for all purposes.

### TECHNICAL FIELD

The present disclosure relates to the fields of cellular and molecular biology, and antibody technology.

### BACKGROUND

Myeloid Derived Suppressor Cell (MDSC)-mediated suppression of immune response has been identified in multiple solid tumors and lymphomas. MDSCs are elevated in advanced colorectal cancer (Toor et al, Front Immunol. 2016; 7:560). MDSCs are also observed in breast cancer, and the percentage of MDSCs in the peripheral blood is increased in patients with later stage breast cancer (Markowitz et al, Breast Cancer Res Treat. 2013 July; 140(1):13-21). MDSC abundance is also correlated with poor prognosis in solid tumors (Charoentong et al, Cell Rep. 2017 Jan. 3; 18(1):248-262).

MDSCs exert suppression over T cells through multiple mechanisms, including the production of reactive oxygen species, nitric oxide, and arginase. These ultimately lead to suppression of DC, NK and T cell activity and increased tumor burden (Umansky et al., Vaccines (Basel) (2016) 4(4):36). MDSCs also contribute to the tumor development and metastasis through the production of soluble factors such as matrix metalloproteinases, VEGF, bFGF, TGF-β and S100A8/A9 which promote neovascularisation, invasion, proliferation and metastasis.

Targeting V-type immunoglobulin domain-containing suppressor of T-cell activation (VISTA), an immune checkpoint molecule expressed primarily on MDSCs, is an attractive therapeutic strategy for removing MDSC-mediated suppression of effector immune cell function.

VISTA-binding antibodies and cells expressing such antibodies are described in WO 2019/185879 A1.

### SUMMARY

In a first aspect, the present disclosure provides a cell of the cell line deposited 7 May 2021 as ATCC Patent Deposit Number PTA-127063.

The present disclosure also provides a population of cells of the cell line deposited 7 May 2021 as ATCC Patent Deposit Number PTA-127063.

The present disclosure also provides a composition comprising a cell or a population of cells according to the present disclosure.

The present disclosure also provides a method of producing an antigen-binding molecule, comprising culturing a cell or a population of cells according to the present disclosure under conditions suitable for expression of the antigen-binding molecule.

In some embodiments, the method comprises:
culturing a cell or a population of cells according to the present disclosure under conditions suitable for expression of the antigen-binding molecule; and
isolating or purifying antigen-binding molecule produced at the preceding step.

The present disclosure also provides a method of producing a pharmaceutical composition, comprising:
culturing a cell or a population of cells according to the present disclosure under conditions suitable for expression of the antigen-binding molecule; and
formulating the antigen-binding molecule produced at the preceding step with a pharmaceutically-acceptable carrier, diluent, excipient or adjuvant.

In some embodiments, the method comprises:
culturing a cell or a population of cells according to the present disclosure under conditions suitable for expression of the antigen-binding molecule;
isolating or purifying antigen-binding molecule produced at the preceding step;
formulating the isolated or purified antigen-binding molecule with a pharmaceutically-acceptable carrier, diluent, excipient or adjuvant.

The present disclosure also provides the use of a cell or a population of cells according to the present disclosure in the production of an antigen-binding molecule which binds specifically to VISTA.

The present disclosure also provides the use of a cell or a population of cells according to the present disclosure in the production of pharmaceutical composition comprising an antigen-binding molecule which binds specifically to VISTA.

The present disclosure also provides an antigen-binding molecule, or a plurality of antigen-binding molecules, obtained by a method according to the present disclosure.

The present disclosure also provides a pharmaceutical composition obtained by a method according to the present disclosure.

The present disclosure also provides an antigen-binding molecule, a plurality of antigen-binding molecules, or a pharmaceutical composition according to the present disclosure, for use in a method of medical treatment or prophylaxis.

The present disclosure also provides an antigen-binding molecule, a plurality of antigen-binding molecules, or a pharmaceutical composition according to the present disclosure, for use in a method of treating or preventing a cancer or an infectious disease.

The present disclosure also provides the use of an antigen-binding molecule, a plurality of antigen-binding molecules, or a pharmaceutical composition according to the present disclosure, in the manufacture of a medicament for use in a method of treating or preventing a cancer or an infectious disease.

The present disclosure also provides a method of treating or preventing a cancer or an infectious disease, comprising administering to a subject a therapeutically- or prophylactically-effective amount of an antigen-binding molecule, a plurality of antigen-binding molecules, or a pharmaceutical composition according to the present disclosure.

In some embodiments in accordance with various aspects of the present disclosure, the cancer is selected from: a cancer comprising cells expressing VISTA, a cancer comprising infiltration of cells expressing VISTA, a cancer comprising cancer cells expressing VISTA, a hematological cancer, leukemia, acute myeloid leukemia, lymphoma, B cell lymphoma, T cell lymphoma, multiple myeloma, mesothelioma, a solid tumor, lung cancer, non-small cell lung carcinoma, gastric cancer, gastric carcinoma, colorectal cancer, colorectal carcinoma, colorectal adenocarcinoma, uterine cancer, uterine corpus endometrial carcinoma, breast cancer, triple negative breast invasive carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, prostate cancer and/or prostate adenocarcinoma.

The present disclosure also provides an antigen-binding molecule, a plurality of antigen-binding molecules, or a pharmaceutical composition according to the present disclosure, for use in a method of treating or preventing a disease in which myeloid-derived suppressor cells (MDSCs) are pathologically-implicated.

The present disclosure also provides the use of an antigen-binding molecule, a plurality of antigen-binding molecules, or a pharmaceutical composition according to the present disclosure, in the manufacture of a medicament for use in a method of treating or preventing a disease in which myeloid-derived suppressor cells (MDSCs) are pathologically-implicated.

The present disclosure also provides a method of treating or preventing a disease in which myeloid-derived suppressor cells (MDSCs) are pathologically-implicated, comprising administering to a subject a therapeutically- or prophylactically-effective amount of an antigen-binding molecule, a plurality of antigen-binding molecules, or a pharmaceutical composition according to the present disclosure.

The present disclosure also provides a method of inhibiting VISTA-mediated signalling, comprising contacting VISTA-expressing cells with an antigen-binding molecule, a plurality of antigen-binding molecules, or a pharmaceutical composition according to the present disclosure.

The present disclosure also provides a method for inhibiting the activity of myeloid-derived suppressor cells (MDSCs), the method comprising contacting MDSCs with an antigen-binding molecule, a plurality of antigen-binding molecules, or a pharmaceutical composition according to the present disclosure.

The present disclosure also provides a method for increasing the number or activity of effector immune cells, the method comprising inhibiting the activity of VISTA-expressing cells with an antigen-binding molecule, a plurality of antigen-binding molecules, or a pharmaceutical composition according to the present disclosure.

The present disclosure also provides an in vitro complex, optionally isolated, comprising an antigen-binding molecule according to the present disclosure bound to VISTA.

The present disclosure also provides a method for detecting VISTA in a sample, comprising contacting a sample containing, or suspected to contain, VISTA with an antigen-binding molecule, a plurality of antigen-binding molecules, or a pharmaceutical composition according to the present disclosure, and detecting the formation of a complex of the antigen-binding molecule with VISTA.

The present disclosure also provides a method of selecting or stratifying a subject for treatment with a VISTA-targeted agent, the method comprising contacting, in vitro, a sample from the subject with an antigen-binding molecule, a plurality of antigen-binding molecules, or a pharmaceutical composition according to the present disclosure, and detecting the formation of a complex of the antigen-binding molecule with VISTA.

The present disclosure also provides the use of an antigen-binding molecule, a plurality of antigen-binding molecules, or a pharmaceutical composition according to the present disclosure, as an in vitro or in vivo diagnostic or prognostic agent.

The present disclosure also provides the use of an antigen-binding molecule, a plurality of antigen-binding molecules, or a pharmaceutical composition according to the present disclosure, in a method for detecting, localizing or imaging a cancer, optionally wherein the cancer is selected from: a cancer comprising cells expressing VISTA, a cancer comprising infiltration of cells expressing VISTA, a cancer comprising cancer cells expressing VISTA, a hematological cancer, leukemia, acute myeloid leukemia, lymphoma, B cell lymphoma, T cell lymphoma, multiple myeloma, mesothelioma, a solid tumor, lung cancer, non-small cell lung carcinoma, gastric cancer, gastric carcinoma, colorectal cancer, colorectal carcinoma, colorectal adenocarcinoma, uterine cancer, uterine corpus endometrial carcinoma, breast cancer, triple negative breast invasive carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, prostate cancer and/or prostate adenocarcinoma.

The present disclosure also provides a kit of parts, comprising: a cell, a population of cells, a composition, an antigen-binding molecule, a plurality of antigen-binding molecules or a pharmaceutical composition according to the present disclosure.

### DESCRIPTION

The present disclosure relates to a cell line having particularly advantageous properties, which expresses an antagonistic VISTA-binding antibody.

### Cells

The present disclosure provides the cell line deposited 7 May 2021 as ATCC Patent Deposit Number PTA-127063. Also provided are cells of the cell line deposited 7 May 2021 as ATCC Patent Deposit Number PTA-127063, and populations of such cells.

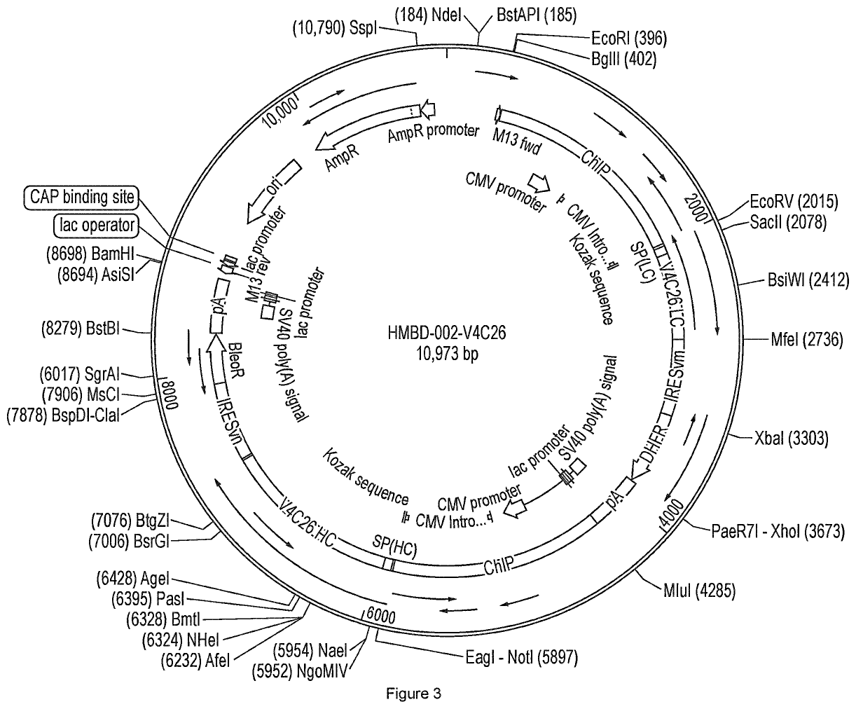

Production of the cell line deposited 7 May 2021 as ATCC Patent Deposit Number PTA-127063 is described in Example 2 herein. The cell line was prepared from cells of the CHO-k1 cell line (ATCC, Cat. No. CCL-61), by electroporation with the polycistronic vector represented schematically in FIG. 3, encoding: (i) V4-C26 hIgG4(S228P) heavy chain, having the amino acid sequence shown in SEQ ID NO:1, and (ii) V4-C26 K light chain, having the amino acid sequence shown in SEQ ID NO:2.

Example 3 describes characterisation of the cell line deposited 7 May 2021 as ATCC Patent Deposit Number PTA-127063. The cell line deposited 7 May 2021 as ATCC Patent Deposit Number PTA-127063 is provided with the following advantageous properties:
High overall productivity, producing secreted V4-C26 hIgG4 at an antibody titer >4.3 g/L in culture at 50 L scale;
High cell-specific productivity (pg/cell/day);
High viability in culture;
Ability to maintain pH in the range of 6.9 to 7.1 without requiring addition of basic species to the culture;
Ability to consume lactate via a lactate consumption metabolic pathway, thereby reducing lactate accumulation in culture;
Produces V4-C26 hIgG4 having a favourable charge variance profile, indicative of high stability, low aggregation propensity and good purification yield by ion exchange chromatography; and
High phenotypic stability, maintaining high overall productivity, high cell-specific productivity, high viability in culture, and production of V4-C26 hIgG4 with a favourable charge variance profile, for at least 59 generations.

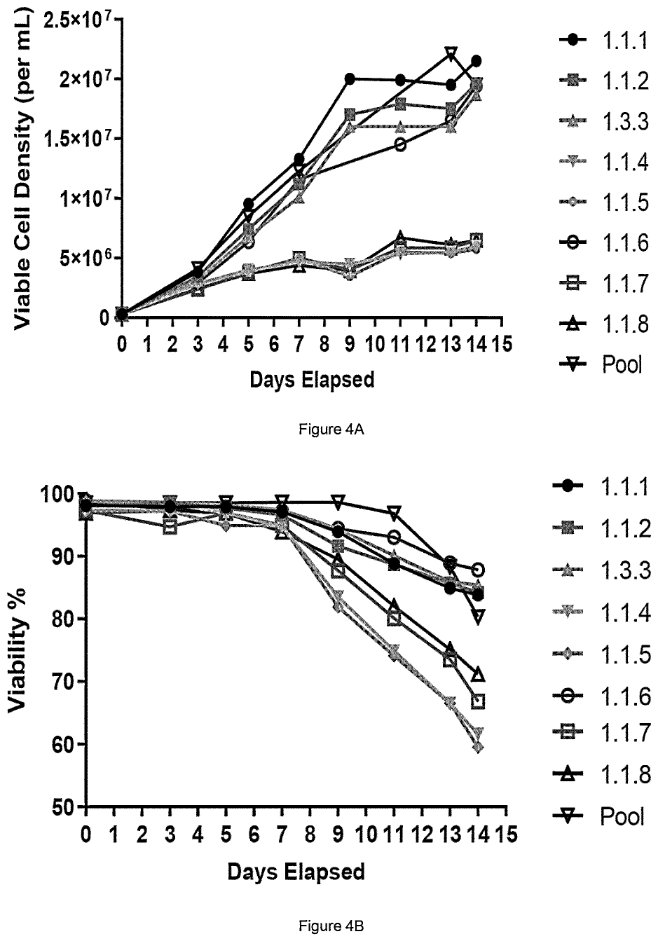

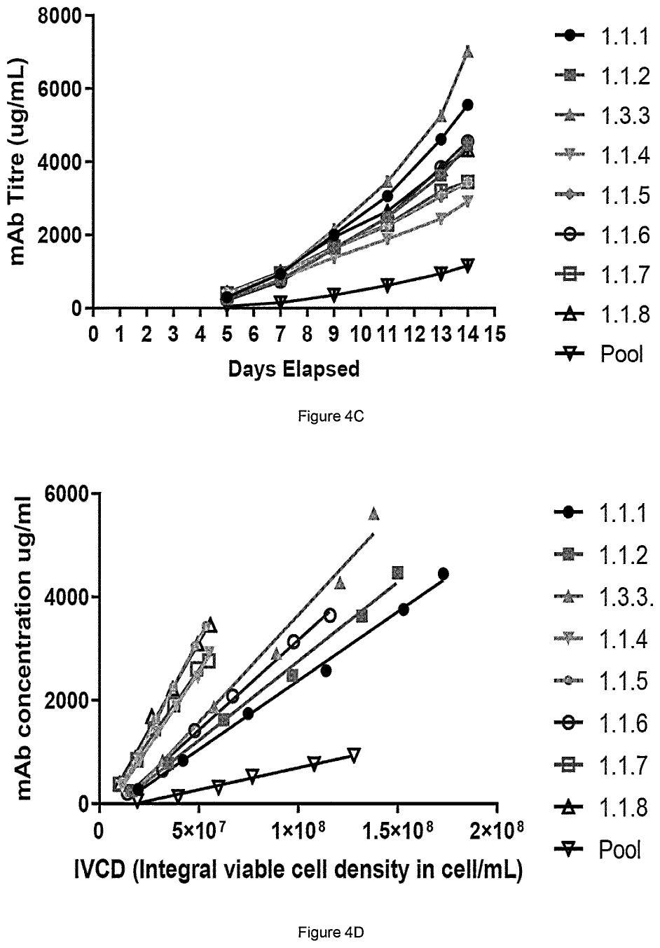

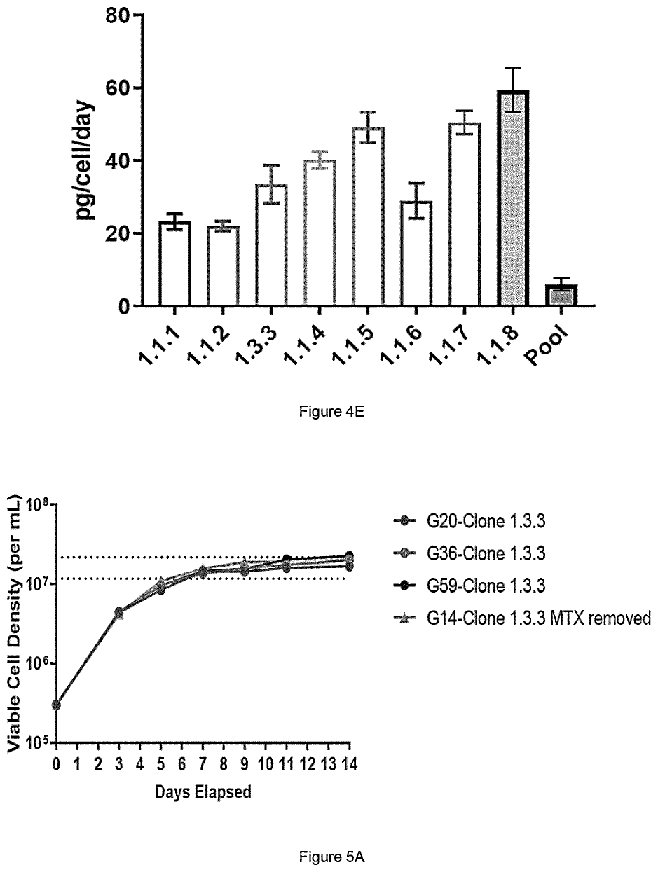

In particular, the cell line deposited 7 May 2021 as ATCC Patent Deposit Number PTA-127063 is demonstrated to produce V4-C26 hIgG4 in culture with a much higher titer than the titer of antibody produced by the stable pool produced by transfection of CHO-k1 cells with HMBD-002-V4C26 (see FIG. 4C), and the cell line also produces V4-C26 hIgG4 with much higher cell-specific productivity than cells of the stable pool (see FIGS. 4D, 4E).

### Methods for Producing Antigen-Binding Molecules and Compositions

The present disclosure provides a methods for producing an antigen-binding molecule, comprising culturing a cell or a population of cells of the cell line deposited 7 May 2021 as ATCC Patent Deposit Number PTA-127063 under conditions suitable for expression of the antigen-binding molecule.

It will be appreciated that the cell line deposited 7 May 2021 as ATCC Patent Deposit Number PTA-127063 comprises nucleic acid encoding the constituent polypeptides of V4-C26 hIgG4 (i.e. the polypeptides of SEQ ID NO:1 and SEQ ID NO:2). Thus, the antigen-binding molecule produced by culturing a cell or a population of cells of the cell line deposited 7 May 2021 as ATCC Patent Deposit Number PTA-127063 under conditions suitable for expression of the antigen-binding molecule is V4-C26 hIgG4.

Suitable culture conditions for the expression of V4-C26 hIgG4 from cells of the cell line deposited 7 May 2021 as ATCC Patent Deposit Number PTA-127063 will be apparent to person skilled in the art. Culture conditions for the expression of monoclonal antibodies from mammalian cells in culture are described e.g. in Birch and Racher, Adv Drug Deliv Rev (2006) 58(5-6):671-85 and Li et al., MAbs (2010) 2(5): 466-477, both of which are hereby incorporated by reference in its entirety. Suitable culture conditions include conditions suitable for the maintenance of cells of the CHO-k1 cell line (ATCC, Cat. No. CCL-61) in in vitro culture.

In some embodiments the culture is performed in culture medium comprising EX-CELL Advanced CHO Fed-Batch Medium (SAFC) or F-12K Medium (ATCC).

In some embodiments, the culture is performed in cell culture medium comprising added L-Glutamine. In some embodiments, the culture is performed in cell culture medium comprising 2 to 12 mM L-Glutamine, e.g. 4 to 10 mM L-Glutamine, e.g. ˜6 mM L-Glutamine.

In some embodiments, the culture is performed in cell culture medium comprising added methotrexate. In some embodiments, the culture is performed in cell culture medium comprising 200 to 300 mM methotrexate, e.g. 225 to 275 mM methotrexate, e.g. ˜250 mM methotrexate. In some embodiments, the culture is performed in the absence of methotrexate.

In some embodiments, the culture is performed in the absence of fetal bovine serum (FBS). In some embodiments, the culture is performed in the absence of human serum. In some embodiments, the culture is performed in the absence of serum. That is, in some embodiments the cells are cultured under ‘serum-free’ conditions.

In particular embodiments, the culture is performed in culture medium comprising EX-CELL Advanced CHO Fed-Batch Medium (SAFC), 2 to 12 mM L-Glutamine (e.g. 4 to 10 mM L-Glutamine, e.g. ˜6 mM L-Glutamine), 200 to 300 mM methotrexate (e.g. 225 to 275 mM methotrexate, e.g. ˜250 mM methotrexate), and in the absence of serum.

It will be appreciated that the culture is performed under suitable environmental conditions, e.g. at 37° C., in 4-10% CO₂ (e.g. 5-8% CO₂), and in a humidified incubator (e.g. at 95% humidity), with our without agitation (e.g. agitation at 75 to 175 rpm, e.g. 100 to 150 rpm, e.g. ˜125 rpm). Culture may be performed in a bioreactor provided with an appropriate supply of nutrients, air/oxygen and/or growth factors.

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

In some embodiments the methods comprise isolating or purifying antigen-binding molecule produced by culturing a cell or a population of cells of the cell line deposited 7 May 2021 as ATCC Patent Deposit Number PTA-127063 under conditions suitable for expression of the antigen-binding molecule.

Following culturing the cells that express the antigen-binding molecules, the expressed antigen-binding molecules may be isolated. Secreted antigen-binding molecules can be collected by partitioning culture media from the cells (e.g. by centrifugation), and isolating/purifying the secreted antigen-binding molecules from the culture media.

Techniques for the isolation/purification of antigen-binding molecules from compositions comprising heterogeneous populations of proteins are well known to those of skill in the art, and are described, for example, in Green and Sambrook, Molecular Cloning: A Laboratory Manual (4th Edition), Birch and Racher, Adv Drug Deliv Rev (2006) 58(5-6):671-85, and Murphy et al., Antibody Technology Journal (2016) 6: 17-32, all of which are hereby incorporated by reference in their entirety.

Large-scale purification of antigen-binding molecules is commonly based on affinity chromatography, and Protein A or G affinity purification is used in many cases. Purification may alternatively, or additionally, comprise purification by anion/cation exchange chromatography, hydrophobic interaction chromatography and/or size exclusion chromatography, which are well known to the person skilled in the art.

The various purification steps are designed to remove contaminant proteins from the cells or culture media to ppm levels, and to reduce DNA to ppb levels. Depending on the processes used, there may be additional specific contaminants to be removed (e.g. leached protein A/G). Purification may comprise filtration (e.g. using a 0.22 μm filter) to remove potential biological contaminants. In addition to contaminants, it may also be necessary to remove undesirable derivatives of the antigen-binding molecule, such as aggregates and degradation products.

In some embodiments, isolating or purifying antigen-binding molecule according to the present disclosure comprises isolation/purification by one or more of: affinity chromatography, (e.g. Protein G chromatography or Protein A chromatography), size exclusion chromatography, high-performance liquid chromatography, ultra-performance liquid chromatography, and ion-exchange chromatography.

Following isolation/purification, the antigen-binding molecule may be provided in a suitable buffer, e.g. for storage. As used herein, a “buffer” refers to a buffered solution that resists changes in pH by the action of its acid-base conjugate components. A composition comprising an antigen-binding molecule according to the present disclosure may comprise the antigen-binding molecule in a buffer.

A buffer of the present disclosure preferably has a pH in the range from about 4.5 to about 7.0, preferably from about 5.0 to about 6.5. Examples of buffers that will control the pH in this range include acetate, histidine, histidine-arginine, histidine-methionine and other organic acid buffers. Antigen-binding molecules may be buffer exchanged into a buffer of interest by buffer dialysis.

In some embodiments, methods of the present disclosure comprise formulating an antigen-binding molecule according to the present disclosure to a composition, e.g. a pharmaceutical composition. In some embodiments the methods comprise mixing an antigen-binding molecule, or mixing a composition comprising an antigen-binding molecule, with a pharmaceutically-acceptable carrier, diluent, excipient or adjuvant.

The present disclosure also provides antigen-binding molecules and compositions produced by the methods described herein.

### Compositions

The present disclosure also provides a composition comprising a cell, or a population of cells, of the cell line deposited 7 May 2021 as ATCC Patent Deposit Number PTA-127063.

The present disclosure also provides an antigen-binding molecule expressed from a cell, or a population of cells, of the cell line deposited 7 May 2021 as ATCC Patent Deposit Number PTA-127063. The present disclosure also provides a composition comprising an antigen-binding molecule according to the present disclosure.

Compositions may comprise an antigen-binding molecule according to the present disclosure provided in a buffer, e.g. a buffer as described herein.

Antigen-binding molecules described herein may be formulated as pharmaceutical compositions or medicaments for clinical use, and may comprise a pharmaceutically-acceptable carrier, diluent, excipient or adjuvant.

Compositions according to the present disclosure may be formulated for topical, parenteral, systemic, intracavitary, intravenous, intra-arterial, intramuscular, intrathecal, intraocular, intraconjunctival, intratumoral, subcutaneous, intradermal, intrathecal, oral ortransdermal routes of administration which may include injection or infusion. Such compositions may comprise the antigen-binding molecule or cell in a sterile or isotonic medium. Medicaments and pharmaceutical compositions may be formulated in fluid, including gel, form. Fluid formulations may be formulated for administration by injection or infusion (e.g. via catheter) to a selected region of the human or animal body. In some embodiments, compositions of the present disclosure may be formulated for injection or infusion, e.g. into a blood vessel or tumor.

### Therapeutic and Prophylactic Applications

The compositions comprising cells of the cell line deposited 7 May 2021 as ATCC Patent Deposit Number PTA-127063, antigen-binding molecules produced from such cells and compositions comprising such antigen-binding molecules find use in therapeutic and prophylactic methods. The therapeutic and prophylactic utility of V4-C26 is evidenced by WO 2019/185879 A1 (which is incorporated by reference in its entirety).

The present disclosure provides an antigen-binding molecule or composition according to the present disclosure for use in a method of medical treatment or prophylaxis. Also provided is the use of an antigen-binding molecule or composition according to the present disclosure in the manufacture of a medicament for treating or preventing a disease or condition. Also provided is a method of treating or preventing a disease or condition, comprising administering to a subject a therapeutically or prophylactically effective amount of an antigen-binding molecule or composition described herein.

The methods may be effective to reduce the development or progression of a disease/condition, alleviate the symptoms of a disease/condition or reduce the pathology of a disease/condition. The methods may be effective to prevent progression of the disease/condition, e.g. to prevent worsening of, or to slow the rate of development of, the disease/condition. In some embodiments the methods may lead to an improvement in the disease/condition, e.g. a reduction in the symptoms of the disease/condition or reduction in some other correlate of the severity/activity of the disease/condition. In some embodiments the methods may prevent development of the disease/condition to a later stage (e.g. a chronic stage or metastasis).

It will be appreciated that the antigen-binding molecules and compositions described herein may be used for the treatment/prevention of any disease/condition that would derive therapeutic or prophylactic benefit from a reduction in the number and/or activity of cells expressing VISTA (e.g. MDSCs). It will also be clear that their therapeutic and prophylactic utility extends to essentially any disease/condition which would benefit from a reduction in the number or activity of MDSCs and/or other cells expressing VISTA, e.g. tumor-associated macrophages (TAMs) and neutrophils. Antagonism of VISTA effectively releases effector immune cells from suppression by MDSCs and/or other cells expressing VISTA.

For example, the disease/condition may be a disease/condition in which cells expressing VISTA (e.g. MDSCs) are pathologically implicated, e.g. a disease/condition in which an increased number/proportion of cells expressing VISTA (e.g. MDSCs) is positively associated with the onset, development or progression of the disease/condition, and/or severity of one or more symptoms of the disease/condition, or for which an increased number/proportion of cells expressing VISTA (e.g. MDSCs), is a risk factor for the onset, development or progression of the disease/condition.

In some embodiments, the disease/condition to be treated/prevented in accordance with the present disclosure is a disease/condition characterised by an increase in the number/proportion/activity of cells expressing VISTA (e.g. MDSCs), e.g. as compared to the number/proportion/activity of cells expressing VISTA (e.g. MDSCs) in the absence of the disease/condition.

In some embodiments, a subject may be selected for treatment described herein based on the detection of an increase in the number/proportion/activity of cells expressing VISTA (e.g. MDSCs), e.g. in the periphery, or in an organ/tissue which is affected by the disease/condition (e.g. an organ/tissue in which the symptoms of the disease/condition manifest), or by the presence of cells expressing VISTA (e.g. MDSCs or tumor-associated macrophages) in a tumor. The disease/condition may affect any tissue or organ or organ system. In some embodiments the disease/condition may affect several tissues/organs/organ systems.

In some embodiments a subject may be selected for therapy/prophylaxis in accordance with the present disclosure based on determination that the subject has an increase in the number/proportion/activity of cells expressing VISTA (e.g. MDSCs) in the periphery or in an organ/tissue relative to the number/proportion/activity of such cells in a healthy subject, or based on determination that the subject has a tumor comprising cells expressing VISTA (e.g. MDSCs).

In some embodiments the disease/condition to be treated/prevented is a cancer.

It will be appreciated that the antigen-binding molecules and compositions described herein are useful for the treatment of cancers in general, because they are useful to release effector immune cells from MDSC-mediated suppression or suppression by cells expressing VISTA, and thereby enhance the anticancer immune response.

The cancer may be any unwanted cell proliferation (or any disease manifesting itself by unwanted cell proliferation), neoplasm or tumor. The cancer may be benign or malignant and may be primary or secondary (metastatic). A neoplasm or tumor may be any abnormal growth or proliferation of cells and may be located in any tissue. The cancer may be of tissues/cells derived from e.g. the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node, lymphoblast, maxilla, mediastinum, mesentery, myometrium, nasopharynx, omentum, oral cavity, ovary, pancreas, parotid gland, peripheral nervous system, peritoneum, pleura, prostate, salivary gland, sigmoid colon, skin, small intestine, soft tissues, spleen, stomach, testis, thymus, thyroid gland, tongue, tonsil, trachea, uterus, vulva, and/or white blood cells.

Tumors to be treated may be nervous or non-nervous system tumors. Nervous system tumors may originate either in the central or peripheral nervous system, e.g. glioma, medulloblastoma, meningioma, neurofibroma, ependymoma, Schwannoma, neurofibrosarcoma, astrocytoma and oligodendroglioma. Non-nervous system cancers/tumors may originate in any other non-nervous tissue, examples include melanoma, mesothelioma, lymphoma, myeloma, leukemia, Non-Hodgkin's lymphoma (NHL), Hodgkin's lymphoma, chronic myelogenous leukemia (CML), acute myeloid leukemia (AML), myelodysplastic syndrome (MDS), cutaneous T-cell lymphoma (CTCL), chronic lymphocytic leukemia (CLL), hepatoma, epidermoid carcinoma, prostate carcinoma, breast cancer, lung cancer, colon cancer, ovarian cancer, pancreatic cancer, thymic carcinoma, NSCLC, hematologic cancer and sarcoma.

MDSCs are elevated in advanced colorectal cancer (Toor et al, Front Immunol. 2016; 7:560). MDSCs are also observed in breast cancer, and the percentage of MDSCs in the peripheral blood is increased in patients with later stage breast cancer (Markowitz et al, Breast Cancer Res Treat. 2013 July; 140(1):13-21). MDSC abundance is also correlated with poor prognosis in solid tumors (Charoentong et al, Cell Rep. 2017 Jan. 3; 18(1):248-262), and MDSCs are enriched in liver cancer models (Connolly et al., J Leukoc Biol. (2010) 87(4):713-25). Prostate and breast carcinomas, melanomas, colorectal cancer and Lewis lung carcinoma have been reported to produce chemokines which attract MDSCs and contribute to immune suppression (Umansky et al., Vaccines (Basel) (2016) 4(4):36)), and MDSCs in pancreatic cancer patients have been positively correlated with tumor burden (Xu et al., Hepatobiliary Pancreat Dis Int. (2016) 15(1):99-105). VISTA has also been reported to be a target for the treatment of ovarian cancer (see e.g. U.S. Pat. No. 9,631,018 B2) and lymphoma (see e.g. WO 2017/023749 A1).

Blando et al. Proc Natl Acad Sci USA. (2019) 116(5):1692-1697 recently reported significant infiltration of VISTA-expressing myeloid cells in pancreatic cancer, and expansion of VISTA-expressing myeloid cells has been observed following treatment with CTLA4 antagonist in prostate cancer, and both pre- and post-treatment with PD-L1 antagonist in melanoma.

In some embodiments, a cancer is selected from: a cancer comprising cells expressing VISTA, a cancer comprising infiltration of cells expressing VISTA, a cancer comprising cancer cells expressing VISTA, a hematological cancer, leukemia, acute myeloid leukemia, lymphoma, B cell lymphoma, T cell lymphoma, multiple myeloma, mesothelioma, a solid tumor, lung cancer, non-small cell lung carcinoma, gastric cancer, gastric carcinoma, colorectal cancer, colorectal carcinoma, colorectal adenocarcinoma, uterine cancer, uterine corpus endometrial carcinoma, breast cancer, triple negative breast invasive carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, prostate cancer and/or prostate adenocarcinoma.

In some embodiments the cancer is colorectal cancer (e.g. colon carcinoma, colon adenocarcinoma), pancreatic cancer, breast cancer, liver cancer, prostate cancer, ovarian cancer, head and neck cancer, leukemia (e.g. T cell leukemia), lymphoma, melanoma, thymoma, lung cancer, non-small cell lung cancer (NSCLC) and/or a solid tumor.

The treatment/prevention may be aimed at one or more of: delaying/preventing the onset/progression of symptoms of the cancer, reducing the severity of symptoms of the cancer, reducing the survival/growth/invasion/metastasis of cells of the cancer, reducing the number of cells of the cancer and/or increasing survival of the subject.

In some embodiments, the cancer to be treated/prevented comprises cells expressing VISTA. In some embodiments, the cancer to be treated/prevented comprises cancer cells expressing VISTA. In some embodiments, the cells expressing VISTA are MDSCs (e.g. g-MDSCs and/or m-MDSCs). In some embodiments, the cancer comprises a tumor comprising cells expressing VISTA (e.g. MDSCs). In some embodiments, the cancer to be treated/prevented comprises a tumor comprising MDSCs. In some embodiments, the cancer to be treated/prevented comprises infiltration of cells expressing VISTA (e.g. MDSCs). In some embodiments, the cancer to be treated/prevented comprises a tumor displaying infiltration of cells expressing VISTA (e.g. MDSCs).

In some embodiments, the cancer to be treated/prevented comprises a tumor comprising a population of CD45+ cells comprising greater than 1%, e.g. ≥2%, ≥5%, ≥10%, ≥15%, ≥20%, ≥25% or ≥30% MDSCs (e.g. as determined by immunoprofiling of the tumor).

In some embodiments, a subject may be selected for treatment described herein based on the detection of a cancer comprising cells expressing VISTA (e.g. MDSCs), or detection of a tumor comprising cells expressing VISTA (e.g. MDSCs), e.g. in a sample obtained from the subject.

In some embodiments the disease/condition in which the VISTA-expressing cells are pathologically implicated is an infectious disease, e.g. bacterial, viral, fungal, or parasitic infection. In some embodiments it may be particularly desirable to treat chronic/persistent infections, e.g. where such infections are associated with T cell dysfunction or T cell exhaustion. It is well established that T cell exhaustion is a state of T cell dysfunction that arises during many chronic infections (including viral, bacterial and parasitic), as well as in cancer (Wherry Nature Immunology Vol. 12, No. 6, p 492-499, June 2011).

Examples of bacterial infections that may be treated include infection by Bacillus spp., Bordetella pertussis, Clostridium spp., Corynebacterium spp., Vibrio cholerae, Staphylococcus spp., Streptococcus spp. Escherichia, Klebsiella, Proteus, Yersinia, Erwina, Salmonella, Listeria sp, Helicobacter pylori, mycobacteria (e.g. Mycobacterium tuberculosis) and Pseudomonas aeruginosa. For example, the bacterial infection may be sepsis or tuberculosis. Examples of viral infections that may be treated include infection by influenza virus, measles virus, hepatitis B virus (HBV), hepatitis C virus (HCV), human immunodeficiency virus (HIV), lymphocytic choriomeningitis virus (LCMV), Herpes simplex virus and human papilloma virus (HPV). Examples of fungal infections that may be treated include infection by Alternaria sp, Aspergillus sp, Candida sp and Histoplasma sp. The fungal infection may be fungal sepsis or histoplasmosis. Examples of parasitic infections that may be treated include infection by Plasmodium species (e.g. Plasmodium falciparum, Plasmodium yoeli, Plasmodium ovale, Plasmodium vivax, or Plasmodium chabaudi chabaudi). The parasitic infection may be a disease such as malaria, leishmaniasis and toxoplasmosis.

The antigen-binding molecules and compositions may exert their therapeutic/prophylactic effects via a molecular mechanism which does not involve an Fc region-mediated effector function (e.g. ADCC, ADCP, CDC). In some embodiments the molecular mechanism does not involve binding of an antigen-binding molecule to an Fcγ receptor (e.g. one or more of FcγRI, FcγRIIa, FcγRIIb, FcγRIIc, FcγRIIIa and FcγRIIIb). In some embodiments the molecular mechanism does not involve binding of an antigen-binding molecule to a complement protein (e.g. C1q).

In some embodiments, the treatment does not induce/increase killing of VISTA-expressing cells. In some embodiments the treatment does not reduce the number/proportion of VISTA-expressing cells.

In some embodiments the treatment (i) inhibits VISTA-mediated signalling, and (ii) does not induce/increase killing of VISTA-expressing cells. In some embodiments the treatment (i) inhibits VISTA-mediated signalling, and (ii) does not reduce the number/proportion of VISTA-expressing cells.

Administration of the antigen-binding molecules and compositions described herein is preferably in a “therapeutically effective” or “prophylactically effective” amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Administration may be alone or in combination with other treatments, either simultaneously or sequentially dependent upon the condition to be treated. The antigen-binding molecule or composition described herein and a therapeutic agent may be administered simultaneously or sequentially.

In some embodiments, the methods comprise additional therapeutic or prophylactic intervention, e.g. for the treatment/prevention of a cancer. In some embodiments, the therapeutic or prophylactic intervention is selected from chemotherapy, immunotherapy, radiotherapy, surgery, vaccination and/or hormone therapy. In some embodiments, the therapeutic or prophylactic intervention comprises leukapheresis. In some embodiments the therapeutic or prophylactic intervention comprises a stem cell transplant.

In some embodiments the antigen-binding molecule or composition described herein is administered in combination with an agent capable of inhibiting signalling mediated by an immune checkpoint molecule other than VISTA. In some embodiments the immune checkpoint molecule is e.g. PD-1, CTLA-4, LAG-3, TIM-3, TIGIT or BTLA. In some embodiments the antigen-binding molecule or composition described herein is administered in combination with an agent capable of promoting signalling mediated by a costimulatory receptor. In some embodiments the costimulatory receptor is e.g. CD28, CD80, CD40 L, CD86, OX40, 4-1 BB, CD27 or ICOS.

Accordingly, the present disclosure provides compositions comprising an antigen-binding molecule or composition according to the present disclosure and an agent capable of inhibiting signalling mediated by an immune checkpoint molecule other than VISTA. Also provided are compositions comprising antigen-binding molecule or composition described herein and an agent capable of promoting signalling mediated by a costimulatory receptor. Also provided is the use of such compositions in methods of medical treatment and prophylaxis of diseases/conditions described herein.

Also provided are methods for treating/preventing diseases/conditions described herein comprising administering an antigen-binding molecule or composition according to the present disclosure and an agent capable of inhibiting signalling mediated by an immune checkpoint molecule other than VISTA. Also provided are methods for treating/preventing diseases/conditions described herein comprising administering an antigen-binding molecule or composition according to the present disclosure and an agent capable of promoting signalling mediated by a costimulatory receptor.

Agents capable of inhibiting signalling mediated by immune checkpoint molecules are known in the art, and include e.g. antibodies capable of binding to immune checkpoint molecules or their ligands, and inhibiting signalling mediated by the immune checkpoint molecule. Other agents capable of inhibiting signalling mediated by an immune checkpoint molecule include agents capable of reducing gene/protein expression of the immune checkpoint molecule or a ligand for the immune checkpoint molecule (e.g. through inhibiting transcription of the gene(s) encoding the immune checkpoint molecule/ligand, inhibiting post-transcriptional processing of RNA encoding the immune checkpoint molecule/ligand, reducing stability of RNA encoding the immune checkpoint molecule/ligand, promoting degradation of RNA encoding the immune checkpoint molecule/ligand, inhibiting post-translational processing of the immune checkpoint molecule/ligand, reducing stability the immune checkpoint molecule/ligand, or promoting degradation of the immune checkpoint molecule/ligand), and small molecule inhibitors.

Agents capable of promoting signalling mediated by costimulatory receptors are known in the art, and include e.g. agonist antibodies capable of binding to costimulatory receptors and triggering or increasing signalling mediated by the costimulatory receptor. Other agents capable of promoting signalling mediated by costimulatory receptors include agents capable of increasing gene/protein expression of the costimulatory receptor or a ligand for the costimulatory receptor (e.g. through promoting transcription of the gene(s) encoding the costimulatory receptor/ligand, promoting post-transcriptional processing of RNA encoding the costimulatory receptor/ligand, increasing stability of RNA encoding the costimulatory receptor/ligand, inhibiting degradation of RNA encoding the costimulatory receptor/ligand, promoting post-translational processing of the costimulatory receptor/ligand, increasing stability the costimulatory receptor/ligand, or inhibiting degradation of the costimulatory receptor/ligand), and small molecule agonists.

Immune suppression by VISTA-expressing MDSCs has been implicated in the failure of, and development of resistance to, treatment with agents capable of inhibiting signalling mediated by an immune checkpoint molecules. Gao et al., Nature Medicine (2017) 23: 551-555 recently suggested that VISTA may be a compensatory inhibitory pathway in prostate tumors after ipilimumab (i.e. anti-CTLA-4 antibody) therapy.

In particular embodiments an antigen-binding molecule or composition according to the present disclosure is administered in combination with an agent capable of inhibiting signalling mediated by PD-1. The agent capable of inhibiting signalling mediated by PD-1 may be a PD-1- or PD-L1-targeted agent. The agent capable of inhibiting signalling mediated by PD-1 may e.g. be an antibody capable of binding to PD-1 or PD-L1 and inhibiting PD-1-mediated signalling. In some embodiments the agent is an antagonist anti-PD-1 antibody. In some embodiments the agent is an antagonist anti-PD-L1 antibody.

In some embodiments, an antigen-binding molecule or composition according to the present disclosure is administered in combination with an agent capable of inhibiting signalling mediated by CTLA-4. The agent capable of inhibiting signalling mediated by CTLA-4 may be a CTLA-4-targeted agent, or an agent targeted against a ligand for CTLA-4 such as CD80 or CD86. In some embodiments, the agent capable of inhibiting signalling mediated by CTLA-4 may e.g. be an antibody capable of binding to CTLA-4, CD80 or CD86 and inhibiting CTLA-4-mediated signalling.

In some embodiments, an antigen-binding molecule or composition according to the present disclosure is administered in combination with an agent capable of inhibiting signalling mediated by LAG-3. The agent capable of inhibiting signalling mediated by LAG-3 may be a LAG-3-targeted agent, or an agent targeted against a ligand for LAG-3 such as MHC class II. In some embodiments, the agent capable of inhibiting signalling mediated by LAG-3 may e.g. be an antibody capable of binding to LAG-3 or MHC class II and inhibiting LAG-3-mediated signalling.

In some embodiments, an antigen-binding molecule or composition according to the present disclosure is administered in combination with an agent capable of inhibiting signalling mediated by TIM-3. The agent capable of inhibiting signalling mediated by TIM-3 may be a TIM-3-targeted agent, or an agent targeted against a ligand for TIM-3 such as Galectin 9. In some embodiments, the agent capable of inhibiting signalling mediated by TIM-3 may e.g. be an antibody capable of binding to TIM-3 or Galectin 9 and inhibiting TIM-3-mediated signalling.

In some embodiments, an antigen-binding molecule or composition according to the present disclosure is administered in combination with an agent capable of inhibiting signalling mediated by TIGIT. The agent capable of inhibiting signalling mediated by TIGIT may be a TIGIT-targeted agent, or an agent targeted against a ligand for TIGIT such as CD113, CD112 or CD155. In some embodiments, the agent capable of inhibiting signalling mediated by TIGIT may e.g. be an antibody capable of binding to TIGIT, CD113, CD112 or CD155 and inhibiting TIGIT-mediated signalling.

In some embodiments, an antigen-binding molecule or composition according to the present disclosure is administered in combination with an agent capable of inhibiting signalling mediated by BTLA. The agent capable of inhibiting signalling mediated by BTLA may be a BTLA-targeted agent, or an agent targeted against a ligand for BTLA such as HVEM. In some embodiments, the agent capable of inhibiting signalling mediated by BTLA may e.g. be an antibody capable of binding to BTLA or HVEM and inhibiting BTLA-mediated signalling.

In some embodiments, methods employing a combination of an antigen-binding molecule or composition according to the present disclosure and an agent capable of inhibiting signalling mediated by an immune checkpoint molecule (e.g. PD-1 and/or PD-L1) provide an improved treatment effect as compared to the effect observed when either agent is used as a monotherapy. In some embodiments the combination of an antigen-binding molecule or composition according to the present disclosure and an agent capable of inhibiting signalling mediated by an immune checkpoint molecule (e.g. PD-1 and/or PD-L1) provide a synergistic (i.e. super-additive) treatment effect.

In some embodiments, treatment with a combination comprising (i) an antigen-binding molecule or composition according to the present disclosure and (ii) an agent capable of inhibiting signalling mediated by an immune checkpoint molecule (e.g. PD-1 and/or PD-L1) may be associated with one or more of:
an improved treatment effect as compared to the treatment effect observed with either component of the combination used alone;
a treatment effect which is synergistic (i.e. super-additive) as compared to the treatment effect observed with either component of the combination used alone;
increased inhibition of tumor growth as compared to inhibition of tumor growth by either component of the combination used alone;
inhibition of tumor growth which is synergistic (i.e. super-additive) as compared to inhibition of tumor growth by either component of the combination used alone;
greater reduction in the number/activity of suppressor immune cells as compared to reduction of the number/activity of suppressor immune cells by either component of the combination used alone;
reduction in the number/activity of suppressor immune cells which is synergistic (i.e. super-additive) as compared to reduction of the number/activity of suppressor immune cells by either component of the combination used alone;
greater reduction of proliferation of suppressor immune cells as compared to reduction proliferation of suppressor immune cells by either component of the combination used alone
reduction of proliferation of suppressor immune cells which is synergistic (i.e. super-additive) as compared to reduction proliferation of suppressor immune cells by either component of the combination used alone;
greater reduction in the proportion of suppressor immune cells within a population of cells (e.g. CD45+ cells, e.g. CD45+ cells obtained from a tumor) as compared to the reduction of the proportion of suppressor immune cells by either component of the combination used alone; and
reduction in the proportion of suppressor immune cells within a population of cells (e.g. CD45+ cells, e.g. CD45+ cells obtained from a tumor) which is synergistic (i.e. super-additive) as compared to the reduction of the proportion of suppressor immune cells by either component of the combination used alone.

Simultaneous administration refers to administration of an antigen-binding molecule or composition according to the present disclosure and therapeutic agent together, for example as a pharmaceutical composition containing both agents (combined preparation), or immediately after each other and optionally via the same route of administration, e.g. to the same artery, vein or other blood vessel.

Sequential administration refers to administration of one of the antigen-binding molecule/composition or therapeutic agent followed after a given time interval by separate administration of the other agent. It is not required that the two agents are administered by the same route, although this is the case in some embodiments. The time interval may be any time interval.

Chemotherapy and radiotherapy respectively refer to treatment of a cancer with a drug or with ionising radiation (e.g. radiotherapy using X-rays or γ-rays). The drug may be a chemical entity, e.g. small molecule pharmaceutical, antibiotic, DNA intercalator, protein inhibitor (e.g. kinase inhibitor), or a biological agent, e.g. antibody, antibody fragment, aptamer, nucleic acid (e.g. DNA, RNA), peptide, polypeptide, or protein. The drug may be formulated as a pharmaceutical composition or medicament. The formulation may comprise one or more drugs (e.g. one or more active agents) together with one or more pharmaceutically acceptable diluents, excipients or carriers.

A treatment may involve administration of more than one drug. A drug may be administered alone or in combination with other treatments, either simultaneously or sequentially dependent upon the condition to be treated. For example, the chemotherapy may be a co-therapy involving administration of two drugs, one or more of which may be intended to treat the cancer.

The chemotherapy may be administered by one or more routes of administration, e.g. parenteral, intravenous injection, oral, subcutaneous, intradermal or intratumoral.

The chemotherapy may be administered according to a treatment regime. The treatment regime may be a pre-determined timetable, plan, scheme or schedule of chemotherapy administration which may be prepared by a physician or medical practitioner and may be tailored to suit the patient requiring treatment.

The treatment regime may indicate one or more of: the type of chemotherapy to administer to the patient; the dose of each drug or radiation; the time interval between administrations; the length of each treatment; the number and nature of any treatment holidays, if any etc. For a co-therapy a single treatment regime may be provided which indicates how each drug is to be administered.

Chemotherapeutic drugs may be selected from: Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), [No Entries], Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib) and Zytiga (Abiraterone Acetate).

Multiple doses of the antigen-binding molecule or composition described herein may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

### Methods of Detection

The present disclosure also provides the antigen-binding molecules or compositions described herein for use in methods for detecting, localizing or imaging VISTA, or cells expressing VISTA (e.g. MDSCs). The antigen-binding molecules described herein may be used in methods that involve binding of the antigen-binding molecule to VISTA. Such methods may involve detection of the bound complex of the antigen-binding molecule and VISTA.

In particular, detection of VISTA may be useful in methods of diagnosing/prognosing a disease/condition in which cells expressing VISTA (e.g. MDSCs) are pathologically implicated, identifying subjects at risk of developing such diseases/conditions, and/or may be useful in methods of predicting a subject's response to a therapeutic intervention.

As such, a method is provided, comprising contacting a sample containing, or suspected to contain, VISTA with an antigen-binding molecule as described herein, and detecting the formation of a complex of the antigen-binding molecule and VISTA. Also provided is a method comprising contacting a sample containing, or suspected to contain, a cell expressing VISTA with an antigen-binding molecule as described herein and detecting the formation of a complex of the antigen-binding molecule and a cell expressing VISTA.

A sample may be taken from any tissue or bodily fluid. The sample may comprise or may be derived from: a quantity of blood; a quantity of serum derived from the individual's blood which may comprise the fluid portion of the blood obtained after removal of the fibrin clot and blood cells; a tissue sample or biopsy; pleural fluid; cerebrospinal fluid (CSF); or cells isolated from said individual. In some embodiments, the sample may be obtained or derived from a tissue or tissues which are affected by the disease/condition (e.g. tissue or tissues in which symptoms of the disease manifest, or which are involved in the pathogenesis of the disease/condition).

Suitable method formats are well known in the art, including immunoassays such as sandwich assays, e.g. ELISA. The methods may involve labelling the antigen-binding molecule, or target(s), or both, with a detectable moiety, e.g. a fluorescent label, phosphorescent label, luminescent label, immuno-detectable label, radiolabel, chemical, nucleic acid or enzymatic label as described herein. Detection techniques are well known to those of skill in the art and can be selected to correspond with the labelling agent.

Methods of this kind may provide the basis of methods for the diagnostic and/or prognostic evaluation of a disease or condition, e.g. a cancer. Such methods may be performed in vitro on a patient sample, or following processing of a patient sample. Once the sample is collected, the patient is not required to be present for the in vitro method to be performed, and therefore the method may be one which is not practised on the human or animal body. In some embodiments the method is performed in vivo.

Detection in a sample may be used for the purpose of diagnosis of a disease/condition (e.g. a cancer), predisposition to a disease/condition, or for providing a prognosis (prognosticating) for a disease/condition, e.g. a disease/condition described herein. The diagnosis or prognosis may relate to an existing (previously diagnosed) disease/condition.

The present disclosure also provides methods for selecting/stratifying a subject for treatment with a VISTA-targeted agent. In some embodiments a subject is selected for treatment/prevention in accordance with the present disclosure, or is identified as a subject which would benefit from such treatment/prevention, based on detection/quantification of VISTA, or cells expressing VISTA, e.g. in a sample obtained from the subject.

Such methods may involve detecting or quantifying VISTA and/or cells expressing VISTA (e.g. MDSCs), e.g. in a patient sample. Where the method comprises quantifying the relevant factor, the method may further comprise comparing the determined amount against a standard or reference value as part of the diagnostic or prognostic evaluation. Other diagnostic/prognostic tests may be used in conjunction with those described herein to enhance the accuracy of the diagnosis or prognosis or to confirm a result obtained by using the tests described herein.

Where an increased level of VISTA is detected, or where the presence of—or an increased number/proportion of—cells expressing VISTA (e.g. MDSCs) is detected in a sample obtained from a subject, the subject may be diagnosed as having a disease/condition in which MDSCs are pathologically implicated, or being at risk of developing such a disease/condition. In such methods, an “increased” level of expression or number/proportion of cells refers to a level/number/proportion which is greater than the level/number/proportion determined for an appropriate control condition, such as the level/number/proportion detected in a comparable sample (e.g. a sample of the same kind, e.g. obtained from the same fluid, tissue, organ etc.), e.g. obtained from a healthy subject.

Where an increased level of VISTA is detected, or where the presence of—or an increased number/proportion of—cells expressing VISTA (e.g. MDSCs) is detected in a sample obtained from a subject, the subject may be determined to have a poorer prognosis as compared to a subject determined to have a lower level of VISTA, or a reduced number/proportion of cells expressing VISTA (e.g. MDSCs) in a comparable sample (e.g. a sample of the same kind, e.g. obtained from the same fluid, tissue, organ etc.).

The antigen-binding molecules and compositions of the present disclosure are also useful in methods for predicting response to immunotherapy. “Immunotherapy” generally refers to therapeutic intervention aimed at harnessing the immune system to treat a disease/condition. Immunotherapy includes therapeutic intervention to increase the number/proportion/activity of effector immune cells (e.g. effector T cells (e.g. antigen-specific T cells, CAR-T cells), NK cells) in a subject. Immunotherapy to increase the number/proportion/activity of effector immune cells includes intervention to promote proliferation and/or survival of effector immune cells, inhibit signalling mediated by immune checkpoint molecules, promote signalling mediated by costimulatory receptors, enhance antigen presentation by antigen-presenting cells, etc. Immunotherapy to increase the number/proportion/activity of effector immune cells also encompasses intervention to increase the frequency of effector immune cells having a desired specificity or activity in a subject e.g. through adoptive cell transfer (ACT). ACT generally involves obtaining immune cells from a subject, typically by drawing a blood sample from which immune cells are isolated. The cells are then typically treated or altered in some way, and then administered either to the same subject or to a different subject. ACT is typically aimed at providing an immune cell population with certain desired characteristics to a subject, or increasing the frequency immune cells with such characteristics in that subject. In some embodiments ACT may e.g. be of cells comprising a chimeric antigen receptor (CAR) specific for a target antigen or cell type of interest. Immunotherapy also includes therapeutic intervention to decrease the number/proportion/activity of suppressor immune cells (e.g. regulatory T cells, MDSCs) in a subject. Immunotherapy to decrease the number/proportion/activity of suppressor immune cells includes intervention to cause or potentiate cell killing of suppressor immune cells, and inhibit signalling mediated by immune checkpoint molecules.

Where an increased level of VISTA is detected, or where the presence of—or an increased number/proportion of—cells expressing VISTA (e.g. MDSCs) is detected in a sample obtained from a subject, the subject may be predicted to have a poorer response to immunotherapy to increase the number/proportion/activity of effector immune cells in the subject as compared to a subject determined to have a lower level of VISTA, or a reduced number/proportion of cells expressing VISTA (e.g. MDSCs) in a comparable sample (e.g. a sample of the same kind, e.g. obtained from the same fluid, tissue, organ etc.). Where an increased level of VISTA is detected, or where the presence of—or an increased number/proportion of—cells expressing VISTA (e.g. MDSCs) is detected in a sample obtained from a subject, the subject may be predicted to have an improved response to immunotherapy aimed at reducing the number/proportion/activity of suppressor immune cells in the subject as compared to a subject determined to have a lower level of VISTA, or a reduced number/proportion of cells expressing VISTA (e.g. MDSCs) in a comparable sample (e.g. a sample of the same kind, e.g. obtained from the same fluid, tissue, organ etc.).

In some embodiments the methods comprise determining the relative size/activity of suppressor immune cell compartment and the effector immune cell compartment. For example, in some embodiments the methods employ the antigen-binding molecules or compositions described herein in methods for determining the ratio of VISTA-expressing cells (e.g. MDSCs, TAMs, neutrophils) to effector immune cells. A subject having an increased ratio may be predicted to have an improved response to immunotherapy aimed at reducing the number/proportion/activity of suppressor immune cells, and/or may be predicted to have a poorer response to immunotherapy to increase the number/proportion/activity of effector immune cells as compared to a subject determined to have a lower ratio.

The diagnostic and prognostic methods of the present disclosure may be performed on samples obtained from a subject at multiple time points throughout the course of the disease and/or treatment, and may be used to monitor development of the disease/condition overtime, e.g. in response to treatment administered to the subject. The results of characterisation in accordance with the methods may be used to inform clinical decisions as to when and what kind of therapy to administer to a subject.

Methods of diagnosis or prognosis may be performed in vitro on a sample obtained from a subject, or following processing of a sample obtained from a subject. Once the sample is collected, the patient is not required to be present for the in vitro method of diagnosis or prognosis to be performed and therefore the method may be one which is not practised on the human or animal body.

### Subjects

The subject in accordance with aspects described herein may be any animal or human. The subject is preferably mammalian, more preferably human. The subject may be a non-human mammal, but is more preferably human. The subject may be male or female. The subject may be a patient. A subject may have been diagnosed with a disease or condition requiring treatment (e.g. a cancer or an infectious disease), may be suspected of having such a disease/condition, or may be at risk of developing/contracting such a disease/condition.

In some embodiments, the subject to be treated according to a therapeutic or prophylactic method of the present disclosure herein is a subject having, or at risk of developing, a cancer. In embodiments according to the present disclosure, a subject may be selected for treatment according to the methods based on characterisation for certain markers of such disease/condition.

### Kits

The present disclosure also provides a kit of parts comprising a cell, or a population of cells, of the cell line deposited 7 May 2021 as ATCC Patent Deposit Number PTA-127063, a composition comprising such a cell or population of cells, antigen-binding molecule(s) expressed from a cell, or a population of cells, of the cell line deposited 7 May 2021 as ATCC Patent Deposit Number PTA-127063, or a pharmaceutical composition comprising such antigen-binding molecule(s). In some embodiments the kit may have at least one container having a predetermined quantity of the relevant article.

In some embodiments, the kit of parts may comprise materials for producing an antigen-binding molecule or composition according to the present disclosure by expression from a cell, or a population of cells, of the cell line deposited 7 May 2021 as ATCC Patent Deposit Number PTA-127063.

In some embodiments, the kit of parts comprises an antigen-binding molecule or composition according to the present disclosure together with instructions for administration to a patient in order to treat a specified disease/condition (e.g. a disease/condition described herein).

In some embodiments the kit of parts may further comprise at least one container having a predetermined quantity of another therapeutic agent (e.g. anti-infective agent or chemotherapy agent). In such embodiments, the kit may also comprise a second medicament or pharmaceutical composition such that the two medicaments or pharmaceutical compositions may be administered simultaneously or separately such that they provide a combined treatment for the specific disease or condition.

### Sequences

| SEQ | | |
|---|---|---|
| ID | | |
| NO: | DESCRIPTION | SEQUENCE |
| 1 | V4-C26 | QVQLQESGPGLVKPSDTLSLTCTVSGYSIT |
| | hIgG4 | SDYTWNWIRQTPGKGLEWIGHITYSGSVSY |
| | (S228P) | NPSLRSRVTISRDTSKNQFSLKLSSVTAAD |
| | HC | TATYSCARALYYPWYFDVWGTGTTVTVSSA |
| | | STKGPSVFPLAPCSRSTSESTAALGCLVKD |
| | | YFPEPVTVSWNSGALTSGVHTFPAVLQSSG |
| | | LYSLSSVVTVPSSSLGTKTYTCNVDHKPSN |
| | | TKVDKRVESKYGPPCPPCPAPEFLGGPSVF |
| | | LFPPKPKDTLMISRTPEVTCVVVDVSQEDP |
| | | EVQFNWYVDGVEVHNAKTKPREEQFNSTYR |
| | | VVSVLTVLHQDWLNGKEYKCKVSNKGLPSS |
| | | IEKTISKAKGQPREPQVYTLPPSQEEMTKN |
| | | QVSLTCLVKGFYPSDIAVEWESNGQPENNY |
| | | KTTPPVLDSDGSFFLYSRLTVDKSRWQEGN |
| | | VFSCSVMHEALHNHYTQKSLSLSLGK |
| 2 | V4-C26 | EIVLTQSPATLSLSPGERATLSCRASSSVG |
| | K LC | YIHWYQQKPGQAPRPIIYTTSNRATGIPAR |
| | | FSGSGSGTDNTLTISSLEPEDSAVYYCQQW |
| | | SSYPPITFGGGTKLEVKRTVAAPSVFIFPP |
| | | SDEQLKSGTASVVCLLNNFYPREAKVQWKV |
| | | DNALQSGNSQESVTEQDSKDSTYSLSSTLT |
| | | LSKADYEKHKVYACEVTHQGLSSPVTKSFN |
| | | RGEC |
| 3 | V4-C26 | CAGGTGCAGCTGCAGGAGTCCGGACCAGGA |
| | hIgG4 | CTGGTGAAGCCAAGCGACACCCTGTCCCTG |
| | (S228P) HC | ACCTGCACAGTGTCCGGCTACTCTATCACA |
| | nucleotide | AGCGATTATACCTGGAACTGGATCAGGCAG |
| | | ACACCTGGCAAGGGACTGGAGTGGATCGGC |
| | | CACATCACATATTCTGGCTCCGTCAGCTAC |
| | | AATCCTTCCCTGCGGTCTAGGGTGACCATC |
| | | TCCAGAGACACATCTAAGAATCAGTTCAGC |
| | | CTGAAGCTGAGCTCAGTGACCGCCGCTGAT |
| | | ACAGCCACGTACAGCTGTGCTCGCGCCCTG |
| | | TACTATCCCTGGTATTTTGACGTGTGGGGC |
| | | ACTGGCACCACAGTGACAGTGTCTAGCGCT |
| | | AGCACCAAGGGCCCATCCGTCTTCCCCCTG |
| | | GCGCCCTGCTCCAGGAGCACCTCCGAGAGC |
| | | ACAGCCGCCCTGGGCTGCCTGGTCAAGGAC |
| | | TACTTCCCCGAACCGGTGACGGTGTCGTGG |
| | | AACTCAGGCGCCCTGACCAGCGGCGTGCAC |
| | | ACCTTCCCGGCTGTCCTACAGTCCTCAGGA |
| | | CTCTACTCCCTCAGCAGCGTGGTGACCGTG |
| | | CCCTCCAGCAGCTTGGGCACGAAGACCTAC |
| | | ACCTGCAACGTAGATCACAAGCCCAGCAAC |
| | | ACCAAGGTGGACAAGAGAGTTGAGTCCAAA |
| | | TATGGTCCCCCATGCCCACCATGCCCAGCA |
| | | CCTGAGTTCCTGGGGGGACCATCAGTCTTC |
| | | CTGTTCCCCCCAAAACCCAAGGACACTCTC |
| | | ATGATCTCCCGGACCCCTGAGGTCACGTGC |
| | | GTGGTGGTGGACGTGAGCCAGGAAGACCCC |
| | | GAGGTCCAGTTCAACTGGTACGTGGATGGC |
| | | GTGGAGGTGCATAATGCCAAGACAAAGCCT |
| | | CGGGAGGAGCAGTTCAACAGCACGTACCGT |
| | | GTGGTCAGCGTCCTCACCGTCCTGCACCAG |
| | | GACTGGCTGAACGGCAAGGAGTACAAGTGC |
| | | AAGGTCTCCAACAAAGGCCTCCCGTCCTCC |
| | | ATCGAGAAAACCATCTCCAAAGCCAAAGGG |
| | | CAGCCCCGAGAGCCACAGGTGTACACCCTG |
| | | CCCCCATCCCAGGAGGAGATGACCAAGAAC |
| | | CAGGTCAGCCTGACCTGCCTGGTCAAAGGC |
| | | TTCTACCCCAGCGACATCGCCGTGGAGTGG |
| | | GAGAGCAATGGGCAGCCGGAGAACAACTAC |
| | | AAGACCACGCCTCCCGTGCTGGACTCCGAC |
| | | GGCTCCTTCTTCCTCTACAGCAGGCTAACC |
| | | GTGGACAAGAGCAGGTGGCAGGAGGGGAAT |
| | | GTCTTCTCATGCTCCGTGATGCATGAGGCT |
| | | CTGCACAACCACTACACACAGAAGAGCCTC |
| | | TCCCTGTCTCTGGGTAAA |
| 4 | V4-C26 KLC | GAGATCGTGCTCACCCAGAGCCCTGCTACA |
| | nucleotide | CTGTCCCTGTCTCCAGGAGAGAGGGCTACC |
| | | CTGTCCTGCAGGGCCAGCTCCTCTGTGGGC |
| | | TACATCCACTGGTATCAGCAGAAGCCAGGA |
| | | CAGGCTCCACGGCCAATTATCTACACCACC |
| | | TCTAACAGAGCTACAGGCATCCCTGCTCGC |
| | | TTCAGCGGCTCCGGTTCTGGCACAGACAAT |
| | | ACCCTGACAATCAGCTCCCTGGAGCCCGAG |
| | | GATTCCGCCGTGTACTATTGTCAGCAGTGG |
| | | TCTAGCTATCCCCCTATCACCTTTGGCGGC |
| | | GGCACAAAGCTGGAGGTGAAGCGTACGGTG |
| | | GCGGCGCCATCTGTCTTCATCTTCCCGCCA |
| | | TCTGATGAGCAGTTGAAATCTGGAACTGCC |
| | | TCTGTTGTGTGCCTGCTGAATAACTTCTAT |
| | | CCCAGAGAGGCCAAAGTACAGTGGAAGGTG |
| | | GATAACGCCCTCCAATCGGGTAACTCCCAG |
| | | GAGAGTGTCACAGAGCAGGACAGCAAGGAC |
| | | AGCACCTACAGCCTCAGCAGCACCCTGACG |
| | | CTGAGCAAAGCAGACTACGAGAAACACAAA |
| | | GTCTACGCCTGCGAAGTCACCCATCAGGGC |
| | | CTGAGCTCGCCCGTCACAAAGAGCTTCAAC |
| | | AGGGGAGAGTGT |

The present disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence is disclosed or referred to herein, the reverse complement thereof is also expressly contemplated.

Methods described herein may preferably performed in vitro. The term “in vitro” is intended to encompass procedures performed with cells in culture whereas the term “in vivo” is intended to encompass procedures with/on intact multi-cellular organisms.

### BRIEF DESCRIPTION OF THE FIGURES

Embodiments and experiments illustrating the principles of the present disclosure will now be discussed with reference to the accompanying figures.

### EXAMPLES

In the following Examples, the inventors describe the generation and characterisation of a novel cell line expressing a VISTA-binding antibody.

FIG. 1. Sensorgrams showing the background-subtracted binding signal for anti-VISTA antibodies V4-C26, 13F3 and VSTB112 to human VISTA and mouse VISTA as determined by Bio-Layer Interferometry.

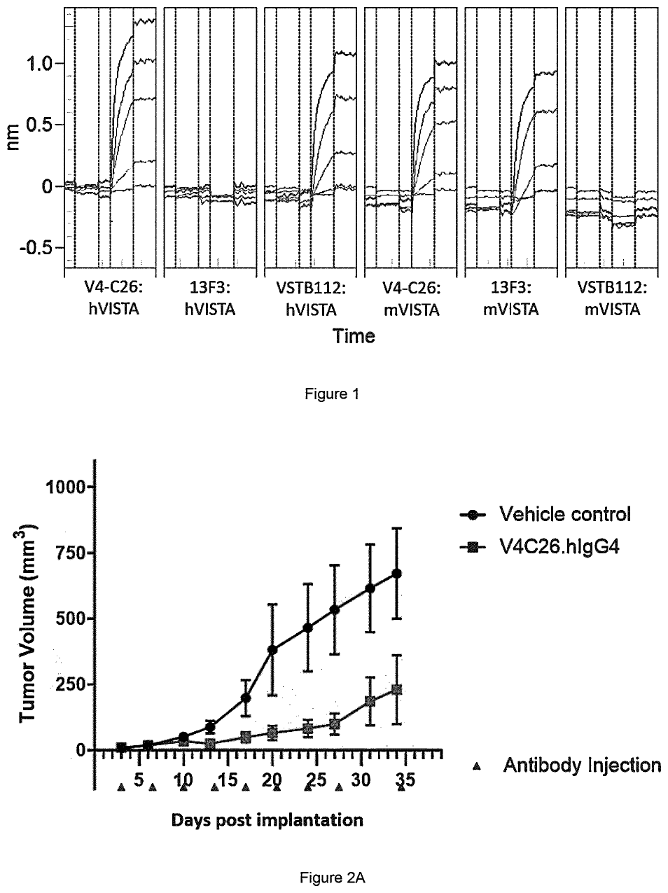

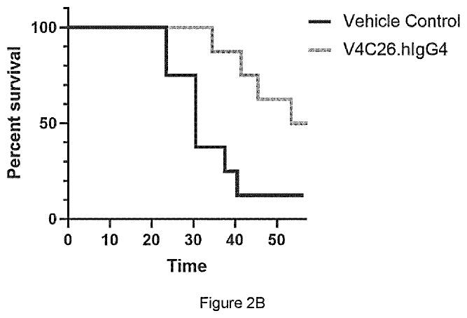

FIGS. 2A and 2B. Graphs showing the effects of administration of V4-C26.hIgG4 on tumor volume and survival of mice in a CT26 cell-derived mouse model of colon carcinoma. (2A) shows tumor volume overtime, and (2B) shows percent survival overtime for mice administered with anti-VISTA antibody V4C26 hIgG4 or vehicle control.

FIG. 3. Schematic representation of the expression vector HMBD-002-V4C26 encoding V4-C26 hIgG4. Sequence features of the vector are described at Example 2.2.

FIG. 4A to 4E. Graphs and bar chart showing (4A) the viable cell density per ml, (4B) the percentage of viable cells in culture, (4C) the monoclonal antibody titer in the cell culture supernatant in μg/ml, (4D) productivity expressed as mAb concentration in μg/ml vs. integral viable cell density in cells/ml, and (4E) cell specific productivity expressed in pg/cell/day (qP), for clones 1.1.1, 1.1.2, 1.3.3, 1.1.4, 1.1.5, 1.1.6, 1.1.7 and 1.1.8, and the stable pool (Pool) produced by transfection of CHO-k1 cells with HMBD-002-V4C26, in a 14-day fed batch process in 45 ml of medium comprising EX-CELL Advanced CHO Fed-Batch Medium+6 mM L-Glutamine+250 nM MTX.

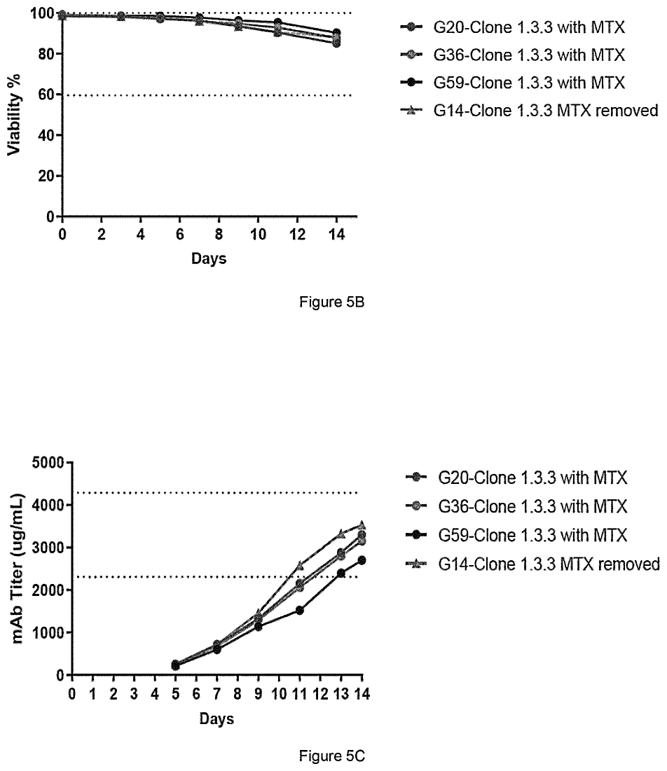

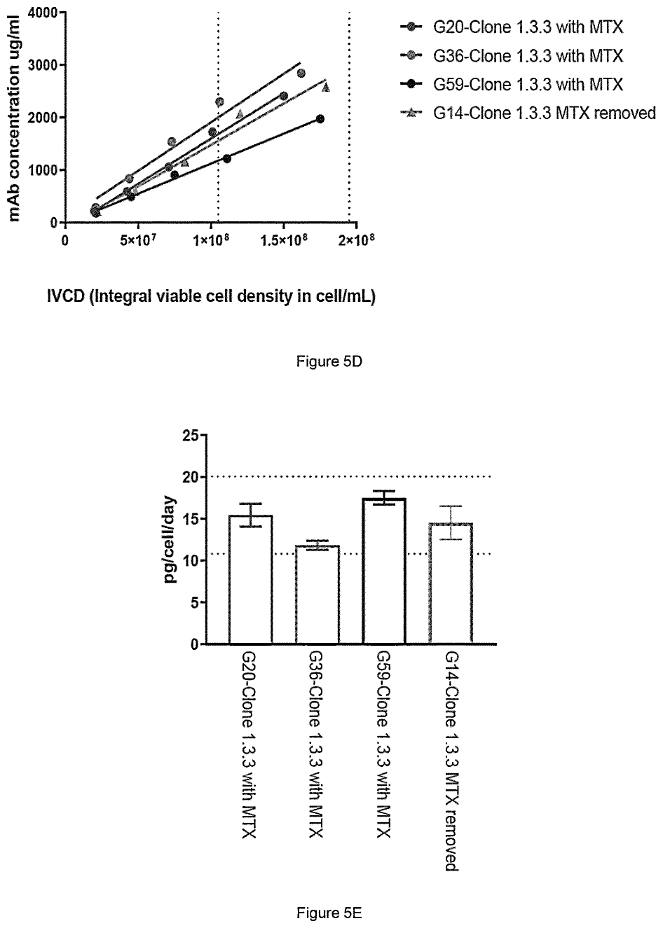

FIG. 5A to 5E. Graphs and bar chart showing (5A) the viable cell density per ml, (5B) the percentage of viable cells in culture, (5C) the monoclonal antibody titer in the cell culture supernatant in μg/ml, (5D) productivity expressed as mAb concentration in μg/ml vs. integral viable cell density in cells/ml, and (5E) cell specific productivity expressed in pg/cell/day (qP), for clone 1.3.3 at generation ˜20 (G20), generation ˜36 (G36), generation ˜59 (G59) and generation ˜14 (G14), in a 14-day fed batch process in 60 ml of medium comprising: (i) EX-CELL Advanced CHO Fed-Batch Medium+6 mM L-Glutamine+250 nM MTX (for G20, G36 and G59), or (ii) EX-CELL Advanced CHO Fed-Batch Medium+6 mM L-Glutamine (for G14).

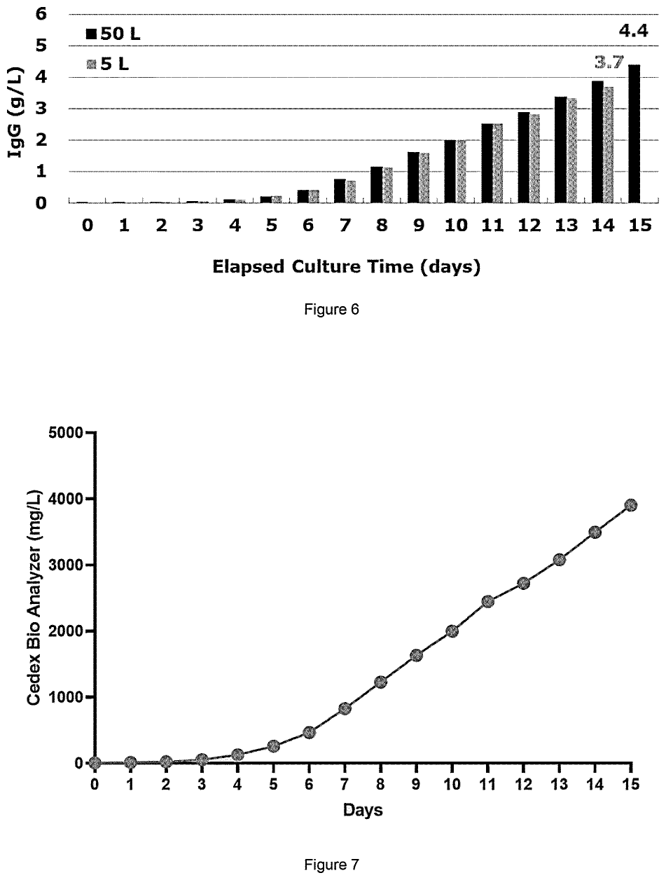

FIG. 6. Bar chart showing monoclonal antibody titer in the cell culture supernatant in g/L at the indicated days, for cells of clone 1.3.3 maintained in bioreactor culture at 5 L or 50 L scale.

FIG. 7. Graph showing monoclonal antibody titer in the cell culture supernatant in mg/L at the indicated days, for cells of clone 1.3.3 maintained in bioreactor culture at 500 L scale.

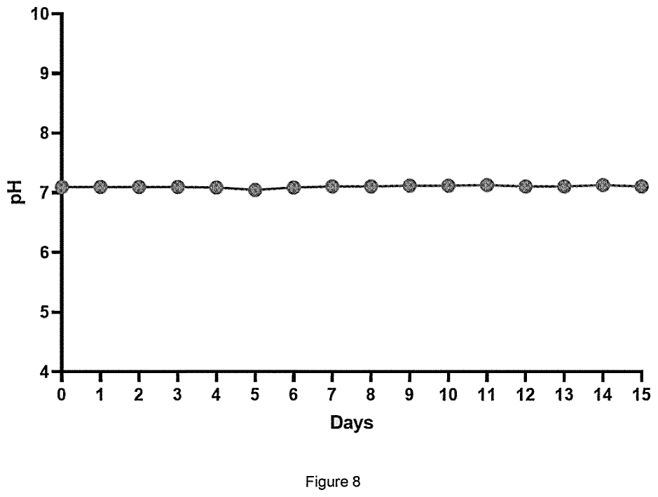

FIG. 8. Graph showing the pH of the culture at the indicated days, for cells of clone 1.3.3 maintained in bioreactor culture at 500 L scale.

### EXAMPLE 1: V4-C26

### 1.1 Characterisation of V4-C26 in WO 2019/185879 A1

The VISTA-binding antibody clone designated V4-C26 is described in WO 2019/185879 A1 (incorporated by reference in its entirety).

V4-C26 comprises the heavy chain variable region shown in SEQ ID NO:289 of WO 2019/185879 A1, and the light chain variable region shown in SEQ ID NO:297 of WO 2019/185879 A1. Example 13 of WO 2019/185879 A1 describes a molecule (molecule [24]) comprising the VH and VL regions of V4-C26 in human IgG1/Vκ format, formed of SEQ ID NO:315 of WO 2019/185879 A1 and SEQ ID NO:317 of WO 2019/185879 A1.

Example 13 and FIG. 53 of WO 2019/185879 A1 show that analysis of V4-C26 using IMGT DomainGapAlign (Ehrenmann et al., Nucleic Acids Res., 38, D301-307 (2010)) and IEDB deimmunization (Dhanda et al., Immunology. (2018) 153(1):118-132) tools revealed that V4-C26 has sufficient homology to human germline heavy and light chains to be considered humanised (i.e. >85%), and numbers of potentially immunogenic peptides few enough to be considered safe and not to present developability issues.

Example 13.1 and FIGS. 45, 46 and 47 of WO 2019/185879 A1 show the results of the analysis of binding of V4-C26 to various different proteins by Biolayer Interferometry. FIGS. 45D, 46B and 47C show that V4-C26 binds to both human VISTA and mouse VISTA with high affinity, and FIG. 45C shows that V4-C26 displays no cross-reactivity with human PD-L1.

Example 13.2 and FIGS. 49, 50, 51, 56 and 57 of WO 2019/185879 A1 show the results of the analysis of binding of V4-C26 to various different proteins by ELISA. FIGS. 49C, 50C and 51C show that V4-C26 has a very low EC₅₀ for binding to human VISTA and mouse VISTA. FIGS. 56C, 57C and 57I show that V4-C26 displays binding to human, mouse, rat and cynomolgous monkey VISTA, yet displays no cross-reactivity for other human B7 protein family members (B7H3, B7H4, B7H6, B7H7, PD-1, CTLA-4).

Example 13.3 and FIGS. 58B and 58C of WO 2019/185879 A1 show that V4-C26 binds to cells expressing human VISTA or mouse VISTA, as determined by flow cytometry.

Example 13.4 and FIG. 52J of WO 2019/185879 A1 show that V4-C26 is thermostable, having a melting temperature of 72.9° C. as determined by Differential Scanning Fluorimetry.

### 1.2 Comparison of Binding to Human and Mouse VISTA for V4-C26, VSTB112 and mAb 13F3

Binding to human VISTA and mouse VISTA was analysed for anti-VISTA antibodies V4-C26 (i.e. molecule [24] of Example 13 of WO 2019/185879 A1), VSTB112 IgG1 (comprising VSTB112 HC (SEQ ID NO: 269 of WO 2019/185879 A1)+VSTB112 LC (SEQ ID NO: 270 of WO 2019/185879 A1)) and mAb 13F3 (BioXCell Cat. No. BE0310).

Bio-Layer Interferometry (BLI) experiments were performed using the Octet QK384 system (ForteBio). All measurements were performed at 25° C.

Briefly, Anti-Penta-HIS (HIS1K) coated biosensor tips (Pall ForteBio, USA) were incubated for 60 sec in PBS buffer (pH 7.2) to obtain a first baseline, and tips were subsequently loaded for 120 sec with 270 nM HIS-tagged human VISTA or mouse VISTA, in PBS (pH 7.2).

After loading, biosensors were incubated for 60 sec in PBS buffer pH 7.2 to obtain a second baseline, and were then incubated for 120 sec with a 4 point, 2 fold dilution series of the test antibodies (concentrations: 250 nM, 125 nM, 62.5 nM and 31.3 nM) in PBS pH 7.2 to obtain association curves. Finally, the biosensors were incubated for 120 sec in PBS pH 7.2 to obtain dissociation curves.

The results are shown in FIG. 1. V4-C26 was found to display binding to both human VISTA and mouse VISTA. By contrast, mAb 13F3 displayed binding to mouse VISTA but not human VISTA, and VSTB112 displayed binding to human VISTA but not mouse VISTA.

1.3 Tumor Growth Inhibition by V4C26 hIgG4

The antigen-binding molecule V4C26 hIgG4 comprising the heavy chain of SEQ ID NO:1 and the light chain of SEQ ID NO:2 was evaluated in a syngeneic cell line-derived mouse model of colon carcinoma for its ability to inhibit tumor growth in vivo.

CT26 cells were obtained from ATCC, and cultured in RPMI-1640 supplemented with 10% fetal bovine serum and 1% Pen/Strep, at 37° C. in a 5% CO₂ incubator.

CT26 cell-derived tumors were established by injecting 1×10⁵ CT26 cells subcutaneously into the right flanks of ˜6-8 week-old female BALB/c mice. 3 days post-implantation, and biweekly thereafter, mice were administered by intraperitoneal injection with 25 mg/kg V4C26 hIgG4, or an equal volume of vehicle as a control condition (8 mice per treatment group).

Tumor volume was measured 3 times a week using a digital caliper, and calculated using the formula [L×W2/2]. The study end point was considered to have been reached once the tumors of the control arm measured >1.5 cm in length.

The results are shown in FIGS. 2A and 2B. Mice administered with V4C26 hIgG4 displayed significant inhibition of tumor growth and improved survival relative to mice administered with vehicle.

### EXAMPLE 2: CELL LINE DEVELOPMENT

The present example describes the production of a cell lines stably expressing antibody V4-C26 hIgG4.

### 2.1 Adaptation to Culture in Serum-Free Medium

CHO-k1 cells (ATCC, Cat. No. CCL-61) were first adapted to suspension culture in serum-free medium. Briefly, CHO-k1 cells were first cultured in F-12K medium supplemented with 10% heat-inactivated FBS (F-12K+10 medium).

After three passages, the medium was exchanged to F75-25 medium (comprising 75% F-12K+10 medium, and 25% “50:50 medium”, “50:50 medium” is medium comprising 50% PF CHO Serum-Free Medium+50% CD CHO Serum-Free Medium+6 mM L-Glutamine+0.05% Pluronic F-68) with a seeding density of 5×10⁵ cell/ml. Cells were transferred to shake flasks and cultured in a 37° C., 8% CO₂, humidified incubator with agitation at 125 rpm.

After eight passages in F75-25 medium, the cell culture was diluted into F50-50 medium (comprising 50% F-12K+10 medium, and 50% 50:50 medium) at seeding density 5×10⁵ cell/ml. The cells were passaged 3 times, and subsequently diluted into F25-F75 medium (comprising 25% F-12K+10 medium, 75% 50:50 medium) at seeding density 5×10⁵ cell/ml.

After three passages, the cells were diluted to F10-90 medium (comprising 10% F-12K+10 medium, 90% 50:50 medium) at seeding density of 5×10⁵ cell/mi and cultured for three passages.

Next, the cells were seeded to a seeding density of 2×10⁵ cell/mi into 100% 50:50 for two passages. At the end of the process of adaption to culture in serum-free medium, the viability of cells in culture was 94.8%.

Cells were further adapted to EX-CELL Advanced CHO Fed-Batch medium (SAFC; Sigma Cat. No. 143660) supplemented with 6 mM L-Glutamine (Sigma Cat. No. G8540), which is hereafter referred to as ‘EX-CELL medium’. Cells were highly adaptable to EX-CELL medium with >99% viability at each passage. For maintenance, cells were diluted in EX-CELL medium at seeding density of 2-3×10⁵ cell/m and cultured at 3700 in an 8% CO₂ atmosphere, humidified incubator with agitation at 125 rpm.

### 2.2 Expression Vector Construction

A polycistronic expression vector encoding SEQ ID NO:1 and SEQ ID NO:2 of V4-C26 hIgG4 was produced by cloning VH and VL region sequences codon-optimised for expression by CHO cells into MabDZ vector (described e.g. in US 2012/0301919 A1).

The polycistronic vector HMBD-002-V4C26 encoding V4-C26 hIgG4 is represented schematically in FIG. 3, and its key sequence features are summarised in the table below.

| Element | Description |
|---|---|
| ChIP | Chimeric promoter consisting of murine cytomegalovirus (CMV) |
| | enhancer (−707 to −286 relative to the transcriptional start site of +1, with |
| | 1 mutation at T-599C) (GenBank: M11788.1) |
| | Spel Restriction Site |
| | Human CMV core promoter consisting of human CMV 1st exon and |
| | human CMV intron A (−223 to +953 relative to the transcriptional start |
| | site of +1, with the following mutations: T-74C, C-70T, G-62A, del211T, |
| | C738G, del740C, C720T, T719G) (GenBank: M60321.1) |
| IRESvm | Mutated encephalomyocarditis virus (EMCV) internal ribosome entry site |
| IRESvn | (IRES) (GenBank: M81861.1) |
| pA | Simian virus 40 (SV40) polyadenylation signal, sequence from |
| | pcDNA3.1/Zeo + vector (Thermo Fisher Scientific) |
| HMBD-002 LC | Antibody light chain cDNA consisting of light chain variable region (VL) |
| | and constant sequence from human IgG kappa chain (CK) (SEQ ID NO: 4) |
| HMBD-002 HC | Antibody heavy chain cDNA consisting of heavy chain variable region |
| | (VH) and constant sequence from human lgG4 comprising S228P in |
| | CH2 (SEQ ID NO: 3) |
| DHFR | Dihydrofolate reductase cDNA (GenBank: NM_010049.3) |
| BleoR | Zeocin resistance gene from pUC57 (GenBank: Y14837.1) |
| Ori. | E. coli origin of replication from pUC57 (GenBank: Y14837.1) |
| AmpR | Ampicilin resistant gene from pUC57 (GenBank: Y14837.1) |
| AmpR promoter | Promoter of Ampicilin resistance gene from pUC57 (GenBank: Y14837.1) |
| Lac promoter | Lac promoter from pUC57 (GenBank: Y14837.1) |
| Lac operator | Lac operator from pUC57 (GenBank: Y14837.1) |

### 2.3 Transfection

Cells were transfected with the vector described in Example 2.2 above.

EX-CELL medium-adapted CHO-k1 cells were thawed and maintained at 37° C., 8% CO₂ humidified incubator, and 125 rpm agitation conditions for one week prior to transfection. 1×10⁷ cells were then seeded at a density of 5×10⁶ cell/ml, and electroporated with 5 μg of linearized expression vector using the 4D-Nucleofector kit (Lonza, Switzerland), electroporation program CA201.

Electroporated cells were incubated at 37° C., 5% CO₂ humidified static cell incubator in 6-well plates containing 2 ml EX-CELL medium for 24 h. Cells where then harvested by centrifugation and resuspended in selection medium comprising EX-CELL Advanced CHO Fed-Batch Medium+6 mM L-Glutamine+250 nM methotrexate (MTX; Sigma Cat. No. M8407)+200 μg/ml Zeocin, at a seeding density of 5×10⁵ cells/ml.

Cells were transferred to fresh selection medium once per week. After four weeks, cells were transferred to maintenance medium (comprising EX-CELL Advanced CHO Fed-Batch Medium+6 mM L-Glutamine+250 nM MTX).

### 2.4 Generation of Stable Clone

The stable clone was generated by three rounds of limiting dilution from the transfected stable pool produced as described in Example 2.3, in medium comprising: 80% EX-CELL CHO Cloning Medium+6 mM L-Glutamine.

Clonality analysis was calculated theoretically using a Poisson distribution. As a result, the probability of getting monoclonal cells after 3 rounds limiting dilution, with first two rounds seeding density at 0.5 cell/well, and last round seeding density at 0.3 cell/well, is 99.9%.

At the final round of limiting dilution, a total of 8 clones were selected for further characterisation: 1.1.1, 1.1.2, 1.3.3, 1.1.4, 1.1.5, 1.1.6, 1.1.7 and 1.1.8.

### EXAMPLE 3: CELL LINE CHARACTERISATION

### 3.1 Growth and Productivity

The cell lines were characterized for growth and productivity by a 14-day fed batch process in 45 ml of medium comprising EX-CELL Advanced CHO Fed-Batch Medium+6 mM L-Glutamine+250 nM MTX.

Viable cell densities, percentage viability, monoclonal antibody titer, and cell-specific productivity were determined.

### Analysis of Viability

Viable cell densities (VCD) and percentage viability were determined by analysis using a Vi-Cell (Beckman Coulter), and using the trypan blue exclusion method. The parameters used were as follows:

| Cell Type | CHO |
|---|---|
| Minimum diameter (microns) | 6 |
| Maximum diameter (microns) | 50 |
| number of images | 50 |
| Aspirate cycles | 1 |
| Trypan blue mixing cycles | 3 |
| Cell brightness (%) | 85 |
| Cell sharpness | 100 |
| Viable cell spot brightness (%) | 75 |
| Viable cell spot are (%) | 5 |
| Minimum circularity | 0 |
| Decluster degree | Medium |
| Instrument calibration | Focus control and |
| | concentration control |

### Analysis of Monoclonal Antibody Titer

Monoclonal antibody titers were determined by quantification using a BLI system (Octet-Protein A), as follows:
Sample preparation
   Cell culture supernatant from Day 5 was diluted 10 times with sterile PBS; giving a total dilution factor of 10
   Cell culture supernatant from Day 7-9 was first diluted 10 times with sterile PBS, followed by a 2 times dilution with 100% assay buffer (10% medium comprising EX-CELL Advanced CHO Fed-Batch Medium with 6 mM L-Gln and 250 nM MTX, with 90% PBS, 0.22 μm filtered); giving a total dilution factor of 20
   Cell culture supernatant from Day 12 was first diluted 10 times with sterile PBS, followed by a 3 times dilution with 100% assay buffer; giving a total dilution factor of 30
   Cell culture supernatant from Day 13 onwards was first diluted 10 times with sterile PBS, followed by a 5 times dilution 100% assay buffer; giving a total dilution factor of 50
Preparation of protein standards
   Purified V4-C26 hIgG4 was diluted in assay buffer to the following concentrations: 1, 3, 10, 30, 100, 300, 500 and 700 μg/ml.
Quantification
   Protein A biosensors were pre-hydrated in assay buffer for 10 min.
   Samples and protein standards were transferred to polypropylene 96-well black flat-bottom plates (Greiner Bio-One) at a volume of 200 μl per well, in duplicate. The samples and standard proteins were then transferred to the Octet QK384 system for titer screening using basic quantification with in-plate standards mode.
   Biosensors were first dipped in sample/standard wells for 120 s to obtain the binding rate of antibody to protein A biosensor. Biosensors were the regenerated with regeneration buffer (10 mM glycine pH 1.7) for 5 s and washed/equilibrated in assay buffer for another 5 s, this regeneration-equilibrium cycle was repeated 4 times before taking measurements for the next sample.
   Two measurements were taken for each sample, and all measurements were performed at 25° C. under 400 rpm agitation.

### Analysis of Cell-Specific Productivity (qP)

Integrated viable cell density (IVCD, cell/ml) between 2 sampling days was calculated using the formula (VCDb+VCDaa)/2×(b−a), where ‘a’ represents cultivation time (in days) at day a, ‘b’ represents cultivation time (in days) at day b, and b>a. ‘VCDb’ is the cell count at day b, and ‘VCDaa’ is the cell count at day a with a dilution factor of 0.9 reflecting the culture replacement by feed medium. IVCD for day 13 is the sum of each interval IVCD.

Integrated titer at day x is calculated by current titer (μg/ml) in addition with all the lost titer during day 4, 6, 8, 11 that occurred before day x. Integrated titer is further corrected for evaporation effects, the rate of which is estimated at 1.333 ml per day due to an observation of 20% (12 ml) lost on the day 14 of Fed-Batch culture, and assuming evaporation rate is constant during the whole Fed-Batch process.

The cell-specific productivity is plotted with mAb titer against IVCD. The qP (pg/cell/day) of day n is calculated using the formula: (integrated titer on day n)/(IVCD on day n)×1×10⁶.

The results are shown in FIGS. 4A to 4E. Clone 1.3.3 had the highest monoclonal antibody titer at day 14, and was highly viable throughout the process.

Clone 1.3.3 displayed the highest cell-specific productivity among clones having an acceptable viable cell density profile.

3.2 Stability Clone 1.3.3 was analysed in order to evaluate its phenotypic stability. Cell growth and productivity characteristics were compared across for different generations of clone 1.3.3 (passaged twice per week with a seeding density of 3×10⁵ cell/ml):
G20: generation ˜20, cultured in the presence of 250 nM MTX;
G36: generation ˜36, cultured in the presence of 250 nM MTX;
G59: generation ˜59, cultured in the presence of 250 nM MTX; and
G14: generation ˜14, cultured in the absence of MTX.

The four different generations were analysed in a 14-day fed batch process in 60 ml of medium comprising EX-CELL Advanced CHO Fed-Batch Medium+6 mM L-Glutamine+250 nM MTX for G20, G36 and G59, or in in 60 ml of medium comprising EX-CELL Advanced CHO Fed-Batch Medium+6 mM L-Glutamine for G14.

The results are shown in FIGS. 5A to 5E. Clone 1.3.3 was found to be highly stable, with high viability high antibody titer and cells-specific productivity maintained at generation 59.

In further experiments, preparations of V4-C26 hIgG4 obtained from the 14 day G20, G36, G59 and G14 batch cultures were analysed by ultra-high performance liquid chromatography cation exchange (UHPLC-CEX) in order to evaluate charge variance.

Briefly, samples of V4-C26 hIgG4 obtained from the cultures were diluted to a concentration of 2 mg/ml in amber glass autosampler vial with water. 5 μL was injected onto a MabPac SCX-10, 5 μm, 4 mm×150 mm column equipped on a Vanquish Flex Binary UHPLC system (Thermo Fisher). IgG variance at different charges were separated using the following instrument method:

| Time (min) | Flow (ml/min) | % A | % B |
|---|---|---|---|
| 0.0 | 0.5 | 95 | 5 |
| 0.5 | 0.5 | 95 | 5 |
| 15.0 | 0.5 | 65 | 35 |
| 18.1 | 0.5 | 65 | 35 |
| 23.0 | 0.5 | 95 | 5 |

Mobile phase A: 1×CX-1, pH Gradient Buffer A (pH 5.6). Mobile phase B: 1×CX-1, pH Gradient Buffer B (pH 10.2). Temperature was controlled at 30° C., UV280 of the flow through was recorded. Acidic variants % was obtained by AUC of total acidic peaks before main peak/AUC of total protein peaks×100%; basic variants % was obtained by AUC of total basic peaks after main peak/AUC of total protein peaks×100%.

The proportions of the acidic variants, basic variants and the main isoform are shown in the table below:

| Clone 1.3.3 | G20 MTX+ | G36 MTX+ | G59 MTX+ | G14 MTX− |
|---|---|---|---|---|
| Acid Variants | 24.36% | 26.73% | 24.14% | 26.6% |
| Basic Variants | 11.21% | 9.83% | 13.82% | 10.96% |
| Main isoform | 64.43% | 63.44% | 62.06% | 63.27% |

The average main isoform was 66.30%±0.01%.

The charge variance profile of antibody V4-C26 hIgG4 produced by clone 1.3.3 was indicative of high stability, low aggregation propensity and good purification yield during antibody purification by ion exchange chromatography.

Cell growth characteristics and productivity were found to be similar across different generations of clone 1.3.3, when cultured in the presence or absence of MTX. A list of key phenotypic characteristics is summarised in the table below:

| Characteristics | G20 MTX+ | G36 MTX+ | G59 MTX+ | G14 MTX− |
|---|---|---|---|---|
| Peak IVCD (cell/ml) | 1.66E7 | 2.0E7 | 2.24E7 | 2.02E7 |
| Titer at day 14 (g/l) by HPLC-ProA | 3.3 g/L | 3.15 g/L | 2.699 g/L | 2.53 g/L |
| IVCD (cell/ml) | 1.5E8 | 1.62E8 | 1.75E8 | 1.79E8 |
| qP (pg/cell/day) | 15.44 | 11.83 | 17.51 | 14.51 |
| Viability at day 14 (%) | 85.1% | 87.8% | 90.3% | 88.2% |
| Main peak by HPLC-SCX | 64.43% | 63.44% | 62.06% | 63.27% |

### 3.3 Bioreactor Culture

Growth and productivity of clone 1.3.3 was characterized in bioreactor culture at 5 L, 50 L and 500 L scale.

Cells of clone 1.3.3 were cultured in medium comprising EX-CELL Advanced CHO Fed-Batch Medium+6 mM L-Glutamine+250 nM MTX, maintained at 37° C., 80% relative humidity, 8% CO₂, with agitation.

Monoclonal antibody titer in the cell culture supernatant of bioreactor culture at 5 L, 50 L and 500 L scale was measured using Cedex Bio Analyzer (Roche), in accordance with the manufacturer's instructions.

The results are shown in FIGS. 6 and 7. The monoclonal antibody titer at day 15 of culture at 50 L and 500 L scale was 4.4 g/L and 3.9 g/L, respectively.

The pH of the 500 L culture was measured throughout culture via inline pH monitoring. FIG. 8 shows that pH of the culture was remarkably stable throughout the 15 day culture period. 3.4 Conclusion Clone 1.3.3 was identified to express V4-C26 hIgG4 with high productivity (>4.3 g/L at 50 L scale).

Clone 1.3.3 was moreover determined to be capable of maintaining pH at range from 6.9 to 7.1 without the need for base addition. This cell line is able to utilise lactate produced as a by-product in culture via a lactate consumption metabolic pathway, with the results that lactate does not accumulate.

A thorough panel of virus safety, sterility and mycoplasma analysis was also performed, and the results were satisfactory.

Clone 1.3.3 was deposited 7 May 2021 as ATCC Patent Deposit Number PTA-127063.

1. A cell of the cell line deposited 7 May 2021 as ATCC Patent Deposit Number PTA-127063.

2. A population of cells of the cell line deposited 7 May 2021 as ATCC Patent Deposit Number PTA-127063.

3. A composition comprising a cell according to claim 1, or a population of cells according to claim 2.

4. A method of producing an antigen-binding molecule, comprising culturing a cell according to claim 1, or a population of cells according to claim 2, under conditions suitable for expression of the antigen-binding molecule.

5. The method according to claim 4, wherein the method comprises:
culturing a cell according to claim 1, or a population of cells according to claim 2, under conditions suitable for expression of the antigen-binding molecule; and
isolating or purifying antigen-binding molecule produced at the preceding step.

6. A method of producing a pharmaceutical composition, comprising:
culturing a cell according to claim 1, or a population of cells according to claim 2, under conditions suitable for expression of the antigen-binding molecule; and
formulating the antigen-binding molecule produced at the preceding step with a pharmaceutically-acceptable carrier, diluent, excipient or adjuvant.

7. The method according to claim 6, wherein the method comprises:
culturing a cell according to claim 1, or a population of cells according to claim 2, under conditions suitable for expression of the antigen-binding molecule;
isolating or purifying antigen-binding molecule produced at the preceding step;
formulating the isolated or purified antigen-binding molecule with a pharmaceutically-acceptable carrier, diluent, excipient or adjuvant.

8. Use of a cell according to claim 1, or a population of cells according to claim 2, in the production of an antigen-binding molecule which binds specifically to VISTA.

9. Use of a cell according to claim 1, or a population of cells according to claim 2, in the production of pharmaceutical composition comprising an antigen-binding molecule which binds specifically to VISTA.

10. An antigen-binding molecule, or a plurality of antigen-binding molecules, obtained by a method according to claim 4 or claim 5.

11. A pharmaceutical composition obtained by a method according to claim 6 or claim 7.

12. An antigen-binding molecule or a plurality of antigen-binding molecules according to claim 10, or a pharmaceutical composition according to claim 11, for use in a method of medical treatment or prophylaxis.

13. An antigen-binding molecule or a plurality of antigen-binding molecules according to claim 10, or a pharmaceutical composition according to claim 11, for use in a method of treating or preventing a cancer or an infectious disease.

14. Use of an antigen-binding molecule or a plurality of antigen-binding molecules according to claim 10, or a pharmaceutical composition according to claim 11, in the manufacture of a medicament for use in a method of treating or preventing a cancer or an infectious disease.

15. A method of treating or preventing a cancer or an infectious disease, comprising administering to a subject a therapeutically- or prophylactically-effective amount of an antigen-binding molecule or a plurality of antigen-binding molecules according to claim 10, or a pharmaceutical composition according to claim 11.

16. The antigen-binding molecule, the plurality of antigen-binding molecules or the pharmaceutical composition for use according to claim 13, the use according to claim 14, or the method according to claim 15, wherein the cancer is selected from: a cancer comprising cells expressing VISTA, a cancer comprising infiltration of cells expressing VISTA, a cancer comprising cancer cells expressing VISTA, a hematological cancer, leukemia, acute myeloid leukemia, lymphoma, B cell lymphoma, T cell lymphoma, multiple myeloma, mesothelioma, a solid tumor, lung cancer, non-small cell lung carcinoma, gastric cancer, gastric carcinoma, colorectal cancer, colorectal carcinoma, colorectal adenocarcinoma, uterine cancer, uterine corpus endometrial carcinoma, breast cancer, triple negative breast invasive carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, prostate cancer and/or prostate adenocarcinoma.

17. An antigen-binding molecule or a plurality of antigen-binding molecules according to claim 10, or a pharmaceutical composition according to claim 11, for use in a method of treating or preventing a disease in which myeloid-derived suppressor cells (MDSCs) are pathologically-implicated.

18. Use of an antigen-binding molecule or a plurality of antigen-binding molecules according to claim 10, or a pharmaceutical composition according to claim 11, in the manufacture of a medicament for use in a method of treating or preventing a disease in which myeloid-derived suppressor cells (MDSCs) are pathologically-implicated.

19. A method of treating or preventing a disease in which myeloid-derived suppressor cells (MDSCs) are pathologically-implicated, comprising administering to a subject a therapeutically- or prophylactically-effective amount of an antigen-binding molecule or a plurality of antigen-binding molecules according to claim 10, or a pharmaceutical composition according to claim 11.

20. A method of inhibiting VISTA-mediated signalling, comprising contacting VISTA-expressing cells with an antigen-binding molecule or a plurality of antigen-binding molecules according to claim 10, or a pharmaceutical composition according to claim 11.

21. A method for inhibiting the activity of myeloid-derived suppressor cells (MDSCs), the method comprising contacting MDSCs with an antigen-binding molecule or a plurality of antigen-binding molecules according to claim 10, or a pharmaceutical composition according to claim 11.

22. A method for increasing the number or activity of effector immune cells, the method comprising inhibiting the activity of VISTA-expressing cells with an antigen-binding molecule or a plurality of antigen-binding molecules according to claim 10, or a pharmaceutical composition according to claim 11.

23. An in vitro complex, optionally isolated, comprising an antigen-binding molecule according to claim 10 bound to VISTA.

24. A method for detecting VISTA in a sample, comprising contacting a sample containing, or suspected to contain, VISTA with an antigen-binding molecule or a plurality of antigen-binding molecules according to claim 10, or a pharmaceutical composition according to claim 11, and detecting the formation of a complex of the antigen-binding molecule with VISTA.

25. A method of selecting or stratifying a subject for treatment with a VISTA-targeted agent, the method comprising contacting, in vitro, a sample from the subject with an antigen-binding molecule or a plurality of antigen-binding molecules according to claim 10, or a pharmaceutical composition according to claim 11, and detecting the formation of a complex of the antigen-binding molecule with VISTA.

26. Use of an antigen-binding molecule or a plurality of antigen-binding molecules according to claim 10, or a pharmaceutical composition according to claim 11, as an in vitro or in vivo diagnostic or prognostic agent.

27. Use of an antigen-binding molecule or a plurality of antigen-binding molecules according to claim 10, or a pharmaceutical composition according to claim 11, in a method for detecting, localizing or imaging a cancer, optionally wherein the cancer is selected from: a cancer comprising cells expressing VISTA, a cancer comprising infiltration of cells expressing VISTA, a cancer comprising cancer cells expressing VISTA, a hematological cancer, leukemia, acute myeloid leukemia, lymphoma, B cell lymphoma, T cell lymphoma, multiple myeloma, mesothelioma, a solid tumor, lung cancer, non-small cell lung carcinoma, gastric cancer, gastric carcinoma, colorectal cancer, colorectal carcinoma, colorectal adenocarcinoma, uterine cancer, uterine corpus endometrial carcinoma, breast cancer, triple negative breast invasive carcinoma, liver cancer, hepatocellular carcinoma, pancreatic cancer, pancreatic ductal adenocarcinoma, thyroid cancer, thymoma, skin cancer, melanoma, cutaneous melanoma, kidney cancer, renal cell carcinoma, renal papillary cell carcinoma, head and neck cancer, squamous cell carcinoma of the head and neck (SCCHN), ovarian cancer, ovarian carcinoma, ovarian serous cystadenocarcinoma, prostate cancer and/or prostate adenocarcinoma.

28. A kit of parts, comprising: a cell according to claim 1, a population of cells according to claim 2, a composition according to claim 3, an antigen-binding molecule or a plurality of antigen-binding molecules according to claim 10, or a pharmaceutical composition according to claim 11.
